(19) United States
(12) Patent Application Publication
(10) Pub. No.: US 2021/0164910 A1
(43) Pub. Date: Jun. 3, 2021

(54) LATERAL FLOW ASSAY DEVICES AND METHOD OF USE

(71) Applicant: Lumos Diagnostics IP Pty Ltd, Box Hill (AU)

(72) Inventors: David JONES, Mentone (AU); Timothy Brett MCDONALD, Hawthorn (AU); Faiyaz TAMBUWALA, Croydon (AU); Joseph Ignatius Maria VAN DER PLAS, Greensborough (AU); India Shaw ELSDON, Solana Beach, CA (US); Geoffrey Charles FIELD, Chelsea Heights (AU); Alina Kaye LOSCHER, Bentleigh East (AU); Say Leong Melvin CHOO, Doncaster (AU); Alexia Isabelle Diane Marie TROLLIET, Hawthorn (AU)

(21) Appl. No.: 17/263,799

(22) PCT Filed: Jul. 29, 2019
(86) PCT No.: PCT/AU2019/000090

§ 371 Date: Jan. 27, 2021

Related U.S. Application Data
(60) Provisional application No. 62/825,492, filed on Mar. 28, 2019.

(30) Foreign Application Priority Data
Jul. 27, 2018 (AU) 2018902733
Nov. 8, 2018 (AU) 2018904261

(51) Int. Cl. G01N 21/84; G01N 33/53

(52) U.S. Cl. CPC G01N 21/8483; G01N 2021/1725; G01N 33/5302

(57) ABSTRACT

The present invention relates to testing biological or industrial samples. Disclosed by preferred embodiments is an electronic assay test reader for reading a lateral flow test strip having a development area comprising a test background region and at least one test result line, the electronic lateral flow assay test reader comprising: a cassette for retaining the test strip and a carrier adapted to removably retain the cassette therein; at least one illumination LED operably associated with one or a combination of the cassette and the carrier for illuminating the test strip, and; a light guide comprising a window structure of one or a combination of the cassette and the carrier to direct light emitted or reflected from a selected portion of the development area of the test strip to a sensor wherein the proportion of the at least one test result line relative to the proportion of test background region in the selected portion of the development area of the test strip is maximised

### RELATED APPLICATIONS

This application claims priority to Australian Provisional Patent Application No. 2018902733, filed in the name of Planet Intellectual Property Enterprises Pty Ltd on 27 Jul. 2018, entitled “Lateral Flow Assay Devices and Method of Use” and, Australian Provisional Patent Application No. 2018904261, filed in the name of Planet Intellectual Property Enterprises Pty Ltd on 8 Nov. 2018, entitled “Lateral Flow Assay Devices and Method of Use” and, U.S. Provisional Patent Application No. 62/825,492, filed in the name of Planet Intellectual Property Enterprises Pty Ltd on 28 Mar. 2019, entitled “Lateral Flow Assay Devices and Method of Use” and, the specifications thereof are incorporated herein by reference in their entirety and for all purposes.

### FIELD OF INVENTION

The present invention relates to the field of testing biological or industrial samples. In a preferred embodiment the present invention relates to the field of diagnostic assays, particularly medical or veterinary diagnostic assays. In particular forms, the invention relates to qualitatively detecting the presence of or quantifying markers in a biological sample. In another form the invention relates to devices, such as cassettes and readers, for detecting results of lateral flow assays. In other forms the invention relates to improving the process of qualitatively detecting the presence of or quantifying markers in a sample. In one particular aspect the present invention is suitable for use as a diagnostic assay for home testing, point of care testing, or laboratory use.

It will be convenient to hereinafter describe the invention in relation to its useful effect in biological assays, however it should be appreciated that the present invention is not so limited and may have other applications, such as for testing for chemical or biological markers in industrial samples.

### BACKGROUND ART

It is to be appreciated that any discussion of documents, devices, acts or knowledge in this specification is included to explain the context of the present invention. Further, the discussion throughout this specification comes about due to the realisation of the inventor and/or the identification of certain related art problems by the inventor. Moreover, any discussion of material such as documents, devices, acts or knowledge in this specification is included to explain the context of the invention in terms of the inventor's knowledge and experience and, accordingly, any such discussion should not be taken as an admission that any of the material forms part of the prior art base or the common general knowledge in the relevant art in Australia, or elsewhere, on or before the priority date of the disclosure and claims herein.

### Lateral Flow Assays

An important field of diagnostics is the use of rapid immunodiagnostic assays to provide speed, accuracy and simplicity in the diagnosis and testing in subjects, such as testing for diseases, conditions, microbes or drugs. A common form of such an assay is a lateral flow immunoassay.

Lateral flow assays are immunoassay based diagnostic tests that are often configured in the form of a test strip of polymeric card to which various testing components are attached. The technology is based on a series of capillary beds, such as pieces of porous paper, microstructured polymer, or sintered polymer, each of which facilitates capillary flow of a liquid sample via capillary action. Reagents are often stored in dry form on various capillary beds. Lateral flow assays can take the form of a sandwich assay or a competitive assay, or in more recent examples, a combination of the two.

In use, a liquid sample, suspected of containing a predetermined analyte or marker, is applied onto a sample pad on the test strip. The sample pad acts as a sponge and holds an excess of the sample fluid. The fluid of the sample then migrates to an adjacent pad, typically named the conjugate pad, which the manufacturer has pre-loaded with reagents, often including a labelled reagent (conjugate). Alternatively, the reagents may be pre-loaded on to the sample pad itself, or mixed with the sample prior to application on to the sample pad. The reagents are rehydrated and interact with the sample and any predetermined analyte or marker, if present in the sample. The reconstituted reagents and sample fluid interact and migrate on to a third capillary bed, often porous nitrocellulose, which has been treated with capture reagents. Finally, the sample fluid enters a final porous material, commonly referred to as the waste pad, which acts as a wick to promote additional capillary act to draw the sample fluid through the lateral flow test and it also acts as a waste container.

In a sandwich type assay, as the sample fluid is drawn along the test strip it allows any of the predetermined analyte or marker that is present to attach to an antibody which has been conjugated to a label, such as colloidal gold, carbon, coloured labelled nanoparticles, fluorescently labelled microparticles or dyes, or enzymes. The labelled analyte is then drawn past a capture region where it attaches to a capture antibody which has been adhered to the material matrix, thus depositing a quantity of the label. Hence, the analyte is “sandwiched” between two antibodies, namely, the labelled antibody and the capture antibody.

In a competitive type assay, as the sample fluid is drawn along the test strip any of the predetermined analyte or marker is involved in competitive binding at the capture region inhibiting the binding of the labelled conjugate to the capture antibody. Thus, the presence of the predetermined analyte or marker results in the absence of the label at the capture region in a competitive assay (a positive test result).

In both sandwich and competitive assays, the capture antibodies are typically placed on the test strip forming a line that can be inspected. Inspection might occur directly by the naked eye for some test devices or indirectly, for example, when an electronic reader is used. Regions of the test strip where there are no capture antibodies are considered the background of the test strip. Lateral flow assays also often comprise a control zone or control line. For a control line, antibodies that bind the labelled conjugate antibodies are placed on the test strip to form a line. The control line is used to confirm that the reagents of the test have rehydrated from the conjugate pad and flown through the test strip, if a control line does not develop or in some cases if it does not meet a certain threshold then the test may be considered invalid, indicating to the user that the test should be repeated.

Lateral flow assay test strips are typically single use, relatively low cost and have low sensitivity compared to other diagnostic assays.

Lateral flow test strips are commonly used for home pregnancy tests which detect the level of the pregnancy hormone human chorionic gonadotropin (hCG) in urine. In recent years, single use electronic tests have been used. The levels of hCG in a pregnant woman's blood and urine rise steeply during the first trimester, and within a few weeks there is a substantial difference in hCG levels between pregnant and non-pregnant women. Thus, the presence of a large amount of hormone biomarker at the time of testing means that the required sensitivity for biomarker detection can be relatively low. In cases where a small concentration of the biomarker needs to be detected, the lack of sensitivity of lateral flow assay test strips may produce result lines that are weak and difficult to detect.

While lateral flow assay test strips have been used in electronic readers in the past, the fields of use are limited. In addition, the types of electronic readers used tend to be electronic bench readers that are restricted to a laboratory or testing location or environment. These bench readers are intended to be used for a large volume of tests and reader cost can be high initially. These readers tend to employ inspection techniques that involve scanning methods, photo-image based or a physical raster scan, to achieve the necessary accuracy, sensitivity and dynamic range.

Very low cost and disposable electronic, lateral flow readers have tended to be restricted to qualitative assays where the positive and negative conditions are well separated or distinguishable and large measurement uncertainty does not detract from the utility of the test. These very low cost electronic readers typically measure the light emission or reflection integrated across a region, where the region includes a test line or control line of interest. If more precise measurement of the strength of the test or control line is needed, then the location of the line within the region and the area of the line relative to the region's area, becomes more critical. Likewise, maximising the relative size of the signal from the line relative to the size of the signal from the entire region becomes critical and hence reducing the signal from the region in comparison with the signal from the line of interest, improves the overall signal to noise ratio of the system and improves the potential sensitivity.

Accordingly, there is a need for an assay method and devices that allow lateral flow assay test strip result lines to be presented in a manner that allows electronic readers to provide reliable, repeatable and accurate results.

There is also an ongoing need to produce assay devices that are low cost, and preferably ultimately disposable, for single use or low volume based testing.

In the past, efforts have been made to address these needs. For example, US patent application publication No. 2003/0017615 (Sidwell et al) teaches the addition of a dye to the lateral flow test strip to increase visual contrast between the developed result line and the background. For example, a typical colloidal gold lateral flow test strip will develop a red-purple result line on a white background. If the background were dyed to be a contrasting colour such as green, the effective visual contrast is increased. This assists visual assessment of the test strip results but may not improve assessment by electronic reader depending on the illumination source (a green background as measured with a green illumination source is effectively the same as a white background) and it requires chemical changes to the test strip which may affect chemical reactions and concomitantly, the accuracy of results.

U.S. Pat. No. 8,445,293 (Babu et al) teaches maximisation of binding analytes and minimisation of non-specific binding by adding a chromatographic carrier to the lateral flow test strip. The carrier reduces non-specific binding in the background region, thereby increasing contrast of the result line. However, this requires changes to test strip chemistry and would incur additional costs.

International (PCT) patent application publication No. WO 2012/099897 (Symbolics, LLC.) relates to lateral flow assays using two dimensional features. Reagents are placed on the lateral flow test strip as dots instead of the traditional line. This creates the ability to print arbitrary shapes instead of the traditional result line. These shapes can be used in the form of words or shapes to increase the perceived contrast of the test and reduce human error or confusion. However, this innovation suffers the drawback that it would require changes to the test strip manufacturing process and would incur additional manufacturing cost. Furthermore, with respect to electronic readers, as there is no actual increase in contrast there would be no significant improvement in readability of the test strip.

U.S. Pat. No. 8,475,731 (Abraham et al) relates to a lateral flow assay reader having a transparent barrier insert to help to accurately align the test strip in the measurement device. However, the transparent insert requires regular cleaning or it will affect the measurement or results. Furthermore, inserting and cleaning the insert are extra process steps that increases complexity and cost of measurement.

U.S. Pat. No. 7,315,378 (Phelan et al) relates to a new optical arrangement for an assay reading device which includes having multiple photodetectors aligned to measure reflection from a single light source. The arrangement has the advantage that fewer light emitters are required for multiple measurement regions, but it also has the disadvantage that a different amount of light will reach each measurement region. The number of parts required leads to a lower cost, but this is at the expense of consistent performance across the measurement regions.

US patent application publication No. 2015/0226752 (Nazareth et al) relates to a device and method for electronic analyte assay wherein multiple light sources are aligned to illuminate a single measurement region. This provides more illumination on each measurement region, but with concomitant need for more light emitters being required for each measurement region. Thus, the increase in measurable signal comes at the cost of additional parts per measurement region.

Chinese patent application publication No. CN104730229 (Wandfo Biotech Co., Ltd.) discloses an electronic reader for a test strip assay detection. The apparatus as described pertains also to a system of multiple light sources with a single corresponding optical detector in the form of a photodetector. However, it is noted that the number of photodetectors is not limited to one and may be two or more, where a plurality of light detectors may receive more reflected signals and help to improve the accuracy of test results. Primarily, the disclosure is directed to an electronic detection device comprising a cassette for accommodating the test strip which has an intersected first light separator and second light separator that is in a T-shaped configuration, wherein the first separator comprises a light source separator and an anti-scatter separator. A plurality of light sources are separated into two groups by the first light source separator at the positions of the light sources. A detection region of the test strip is separated from a blank region by the second anti-scatter separator. The light sources are separated from a light detector by the second separator. The second anti-scatter separator does not contact the light detector so as to form a first transmitting gap. The second separator does not contact the test strip so as to form a second transmitting gap and rays reflected from the detection region and the blank region can sequentially penetrate through the second transmitting gap and the first transmitting gap and enter the light detector to be detected. Accordingly, the photoelectric detection device is capable of effectively preventing light interference and the accuracy of the detection result may be significantly improved.

U.S. Pat. No. 9,243,997 (Petruno et al) relates to a lateral flow assay system and method in which multiple measurements of subsections of the measurement region are taken. This scanning arrangement optimises reading of the result line by ensuring that only the relevant signal is analysed and all the background can be discarded. However, it requires an array of measurement sensors or moving parts so that the complexity, cost of parts and assembly costs of the scanning device is much higher than any static reader.

As noted in international (PCT) patent application publication No. WO 2011/048381 (SPD Swiss Precision Diagnostics, GmbH) the trend towards digitally-read devices aims to remove any element of interpretation of the result needed by the user or medical professional. These devices may be two-piece kits, the test strip being incorporated in one type of assay device such as a test stick, which is inserted into a cavity (“test bay”), as described by WO 2011/048381, of a separate reader to digitally read the assay result via optical or other reading elements. The test stick is generally a low cost, disposable element, whereas the reader is more sophisticated and may be reusable. In such kits, it is generally important to ensure that the appropriate regions of the test strip are correctly aligned with the reading elements. An extremely high level of precision of positioning is desired to maximise accuracy, especially when the assay results in the appearance of, or change in, one or more thin lines on the test strip which must be detected by the reading elements. Desirably, therefore, the kit should include features which guarantee accurate positioning of the test strip each time, even when used by an unskilled user. Accordingly, WO 2011/048381 discloses a connection assembly for a test device comprising a carriage for receiving at least a portion of a test device and a receptacle for co-operation with the carriage. The carriage is longitudinally movable with respect to the receptacle and is latchable to the receptacle at a predetermined ‘pre-reading’ position. Whilst there is brief mention of non-magnetic latching means in the form of a sprung pin or other common means known at the time, this prior art disclosure is directed towards the reader comprising magnetic means for latching the assay device onto the reader within the cavity at a predetermined reading position, said latching either being direct latching or via latching of the carriage onto the reader.

In another example mentioned in the preamble of WO 2011/048381, European patent publication No. EP0833145 discloses a “lock and key” location feature and combined switch actuation mechanism, that is provided inside a test bay which engages with a corresponding mating feature on the test stick. The test bay is formed by two case halves, one half being slidable and acting as a carriage to guide the test stick gently into position with the assistance of runners and an elastic band, upon application of a linear insertion force by the user. The carriage releasably clicks into place on the other case half when the test stick has been inserted the correct distance and the location features are engaged. This design is considered to be preferred for applications in which the reader is used only once or only a limited number of times, such as for pregnancy tests or ovulation tests. Wear of the device is not a major problem, but there is room for improvement in terms of the precision positioning desired, because it is subject to problems caused by slight manufacturing variations.

Further examples of prior art electronic lateral flow assay test devices and readers are as follows.

U.S. Pat. No. 9,807,543 (Zin et al) discloses a test device configured for wireless communication of the initiation of a test and wireless communication and data transfer of test results. The invention disclosed within this reference is directed to expanding the usefulness of hand-held or portable test kits, particularly with respect to data communications.

US patent application publication No. US 2016/0202190 (Hein et al) discloses an improved camera imaging technique for lateral flow assay tests, which is intended for increasing the speed of obtaining test results.

US patent application publication No. US 2010/0172802 (Sharrock et al) discloses a device for determining a test result based in part on detecting the flow rate of an analyte on a lateral flow assay test strip. The device includes a light detection system for detecting light reflected from first and second zones of the test strip including a signal indicative of an amount of analyte present and a processor for determining a result indicative of the time required for sample analyte to flow from the first zone to the second zone.

US patent application publication No. US 2015/0094227 (McCarthy et al) discloses a single-use pregnancy test device directed to an improved assay for detecting pregnancy by use of a combined measurement for hCG (human chorionic gonadotrophin), FSH (follicle-stimulating hormone) and a progesterone metabolite.

US patent application publication No. US 2016/0139156 (Lakdawala) discloses a multi-use lateral flow assay test strip reader for ovulation and pregnancy. The disclosure is primarily directed to the flexibility in operation of a base reader with different sensing heads including a lateral flow/colour change reader and a basal temperature sensing cassette.

US patent application publication No. US 2012/0021531 (Ellis et al) discloses a single-use lateral flow assay test reader for determining an estimate of the length of time since conception for a pregnancy test. The disclosure of the test reader is primarily directed to a comparison of assays to a stored analyte threshold for measuring levels of hCG over an extended analyte range. The reader itself as disclosed includes a first assay flow-path having a detection zone for measuring hCG in a lower concentration range and a second assay flow-path having a detection zone for measuring hCG in a higher concentration range. The assay device may include a shared reference zone, a shared control zone and each flow-path may comprise a single detection zone. It further includes a single light detector to detect light from both detection zones and four light sources to respectively illuminate the shared reference zone, the shared control zone and the two detection zones.

US patent application publication No. US 2012/0021531 (Ellis et al) discloses an in vivo immunoassay device for insertion to a patient's body in the form of an autonomous swallowable capsule where a chromatography strip for immunoassay of a body lumen substance is provided along with a sensor to sense a property of the chromatography strip.

U.S. Pat. No. 9,488,585 (Emeric et al) discloses a multi-use optical and electrochemical assay test reader. The disclosed system is adapted to read both a lateral flow and an electrochemical test on the same device. For detection, a camera reader is utilised for the lateral flow assay test.

US patent application publication No. US 2009/0155921 (Lu et al) discloses a multi-use lateral flow assay test reader. The disclosure is primarily directed to a scanning method in which a spring arrangement with a damper for speed control is used to transport or scan the test strip past a measurement sensor.

US patent application publication No. US 2012/0321519 (Brown) also discloses a multi-use lateral flow assay test reader and more specifically a connection assembly for an assay test device. The disclosure is directed to providing accurate positioning of a cassette in a reader using magnets & other mechanical features. The connection assembly comprises a carriage for receiving at least a portion of a test device and a receptacle for co-operation with the carriage where the carriage is longitudinally movable with respect to the receptacle and is latchable to the receptacle at a predetermined position. The reader comprises magnetic means for latching the assay test device onto the reader within said cavity at a predetermined reading position. The latching is either direct latching or via latching of the carriage onto the reader.

The preceding discussion of background art is intended to facilitate an understanding of the present invention only. The discussion is not an acknowledgement or admission that any of the material referred to is or was part of the common general knowledge as at the priority date of the application.

### SUMMARY OF INVENTION

It is an object of preferred embodiments described herein to provide an electronic reader for lateral flow assay test strips.

It is an object of the embodiments described herein to overcome or alleviate at least one of the above noted drawbacks of prior art systems or to at least provide a useful alternative to prior art systems.

In one aspect of embodiments the invention provides an electronic lateral flow assay test reader for reading a lateral flow test strip, the electronic lateral flow assay test reader having a light guide comprising a window structure for framing a development area of the test strip, the development area comprising portions that include a test background region and at least one test result line, wherein the dimensions of the window structure are configured to maximise the proportion of the at least one test result line framed relative to the proportion of test background region framed.

The window structure preferably comprises individual windows for framing respective portions of the development area of the test strip such that any of the test background region framed by the window structure is minimised.

In preferred embodiments the test strip includes strip background and the window structure further comprises at least one window for framing strip background.

Preferably, the respective portions of the development area of the test strip framed by the individual windows comprises one or more of:

a test line;

a control line.

The reader has a housing which may be of at least two parts which alone or in combination retain reader components including:

the test strip;

a PCB incorporating test measurement components; and

the light guide as a separate element.

The light guide may be disposed in close proximity to the test strip.

In embodiments the electronic reader may further comprise a carrier adapted to retain reader components including a removably insertable cassette adapted for containing the lateral flow test strip.

In a preferred embodiment of the present invention there is provided an electronic lateral flow assay test reader for reading a lateral flow test strip, the electronic lateral flow assay test reader having a light guide comprising a window structure for framing a development area of the test strip, the development area comprising portions that include a test background region and at least one test result line, or result line(s) wherein the dimensions of the window structure are configured to maximise the proportion of the at least one test result line framed relative to the proportion of test background region framed and wherein the electronic lateral flow assay test reader is characterised by the window structure comprising individual windows for framing respective portions of the development area of the test strip such that any of the test background region framed by the window structure is minimised.

In a preferred embodiment the electronic reader comprises a unitary housing for releasably receiving and engaging with the carrier.

The window structure of the light guide may be formed by one or a combination of:

the carrier;

the cassette.

The electronic reader may further comprise:

illumination sources for illuminating the at least one test result line and the test background region of the development area of the lateral flow test strip, and;

measurement sensors for detecting light received from the at least one test result line.

Preferably, each respective illumination source is paired with each respective measurement sensor.

Preferably the cassette comprises:

a recess for receiving and nesting the lateral flow test strip therewithin,

at least two or more windows for framing respective portions of the development area of the test strip, the dimensions of the window being configured to maximise the proportion of at least one result line framed relative to the proportion of test background framed.

In preferred embodiments surfaces of the cassette comprise minimally reflective material.

In another aspect of embodiments, the invention provides an electronic reader for a lateral flow assay test strip, the electronic reader comprising:

a recess for receiving and nesting the lateral flow assay test strip therein;

at least one LED illumination source for illuminating one or more result lines or a test background region on the test strip; and

at least one illumination sensor for sensing illumination reflected from the one or more result lines on the test strip,

wherein a current of electricity supplied to each LED illumination source is measured for detecting changes in temperature and changes in LED supply voltage during illumination of the lines on the test strip, and the changes used to calculate applied compensation.

Preferably, the compensation is calculated and applied by measuring the forward current prior to the start of the test, and then again after the sample has developed and the test strip is ready to measure. Furthermore, the difference in the forward currents as a ratio may be calculated in a software routine and used to compensate for temperature and voltage effects which influence the forward current between the start of the test and when the sample is ready. The electronic reader may be operably associated with a voltage source arrangement used to power the at least one LED.

In a further aspect of embodiments, the invention provides an electronic reader for a lateral flow assay test strip, the electronic reader comprising:

a cassette for receiving and nesting the lateral flow assay test strip therein;

a PCB operatively associated with a light guide and including;

at least one LED illumination source for illuminating test and control lines and test background regions on the test strip, and

at least one illumination sensor for sensing illumination received from the lines on the test strip,

wherein one or more of the cassette and the PCB of the reader are adapted for engagement with a unitary housing of the reader.

In another aspect of embodiments, the invention provides apparatus for an electronic reader of a lateral flow assay test strip, the apparatus comprising:

a cassette comprising a recess for receiving and nesting the lateral flow assay test therewithin;

at least one LED illumination source for illuminating result lines and test background regions on the test strip, and;

illumination sensors for sensing illumination received from the result lines on the test strip,

wherein the cassette is removably retained within the reader by a retention mechanism.

In preferred embodiments the retention mechanism is formed by parts of one or a combination of the reader, the cassette and a carrier accommodating the cassette for engagement with the reader and the retention mechanism is adapted to align individual windows of one or a combination of the cassette and the carrier wherein the aligned windows frame respective portions of a development area of the test strip.

The retention mechanism may comprise a snap fit mechanism residing upon or within the cassette and/or the reader including one or more of:

snap fingers for retaining the cassette in place within the reader, and;

biasing means which assists in releasing the cassette from the reader,

which are adapted to work together to ensure that the cassette is positioned consistently and correctly in the reader.

Preferably, the snap fingers reside on the cassette and the biasing means resides on the carrier or the reader.

Preferably, the biasing means comprises leaf springs that urge the cassette towards the electronic components of the reader used for measuring.

In a preferred embodiment, the reader comprises a self-closing door that prevents contaminants from entering a cavity of the multiuse reader when a cassette is not installed in the multiuse reader. The door acts to align the cassette within the reader.

The retention mechanism described herein may further comprise retention clips that are operatively associated with the light guide.

An alignment pin may be provided for engaging one or more of:

the reader;

the light guide;

the cassette;

the carrier.

Preferably, the reader is operable with the cassette by one of:

a slide-on mechanism; or

a clip-on mechanism.

In another aspect of embodiments, the invention provides an electronic reader for a lateral flow assay, the electronic reader comprising,

a recess for receiving and nesting a lateral flow assay test strip therein;

at least one LED illumination source for illuminating one or more result lines and test background regions on the test strip, and illumination sensors for sensing illumination received from the one or more result lines on the test strip;

input/output (IO) pins wherein each pin is operatively associated with two or more LEDs of the reader.

A combination of charlieplexing and multiplexing may be used to control the two or more LEDs. The two or more LEDs may be controlled from five digital IO pins. In preferred embodiments, only a single LED is powered at once.

Further, the reader may be adapted to detect the presence/absence of a cassette containing the lateral flow assay test strip. Moreover, the reader may be adapted to detect the presence/absence of a cassette containing the lateral flow assay test strip using the LEDs and sensors and one or more threshold signals detected where a first measured signal corresponds to a cassette is present and a second measured signal corresponds to a cassette is not present.

In another aspect of embodiments, the invention provides a lateral flow assay test system comprising an electronic reader as disclosed herein or the apparatus as disclosed herein.

In yet another aspect of embodiments the invention provides a method of assessing result lines of a lateral flow assay test strip comprising the steps of:

inserting the assay test strip into an electronic reader as disclosed herein or the apparatus as disclosed herein; and

initiating the illumination source of the electronic reader and detecting illumination received from result lines on the assay test strip.

In still another aspect of embodiments the invention provides an electronic lateral flow assay test reader for reading a lateral flow test strip having a development area, the development area comprising portions that include a test background region and at least one test result line, the electronic lateral flow assay test reader comprising:

a cassette for retaining the test strip and a carrier adapted to removably retain the cassette therein;

at least one illumination LED operably associated with one or a combination of the cassette and the carrier for illuminating the test strip, and;

a light guide comprising a window structure to direct the light emitted from the at least one illumination LED to a selected portion of the development area of the test strip, wherein the window structure is formed by:

one of the cassette or the carrier, or,

a combination of the cassette and the carrier so as to split the light guide between the cassette and the carrier.

The electronic reader may be further characterised by the window structure of the light guide framing the development area of the test strip by the dimensions of the window structure being configured to maximise the proportion of the at least one test result line framed relative to the proportion of test background region framed.

The electronic reader may also be further characterised by the window structure comprising individual windows for framing respective portions of the development area of the test strip such that any of the test background region framed by the window structure is minimised.

In preferred embodiments of the electronic reader a shallow recess is provided between windows of the cassette and the carrier to avoid direct contact therebetween.

In yet another aspect of embodiments the invention provides an electronic lateral flow assay test reader for reading a lateral flow test strip having a development area comprising a test background region and at least one test result line, the electronic lateral flow assay test reader comprising:

a cassette for retaining the test strip and a carrier adapted to removably retain the cassette therein;

at least one illumination LED operably associated with one or a combination of the cassette and the carrier for illuminating the test strip, and;

a light guide comprising a window structure of one or a combination of the cassette and the carrier to direct light emitted or reflected from a selected portion of the development area of the test strip to a sensor wherein the proportion of the at least one test result line relative to the proportion of test background region in the selected portion of the development area of the test strip is maximised.

In yet another aspect of embodiments the invention provides a cassette suitable for a lateral flow assay electronic reader, the cassette comprising,

a recess for receiving and/or nesting a lateral flow test strip,

at least one window for framing a development area of the test strip when nested in the recess, the dimensions of the window being configured to maximise the proportion of at least one test result line of the development area framed relative to the proportion of a test background region of the development area framed,

wherein the surfaces of the cassette comprise minimally reflective material.

In yet another aspect of embodiments the invention provides an electronic reader for a lateral flow assay test strip, the electronic reader comprising,
an opening for receiving the lateral flow assay test strip, preferably a cassette containing the lateral flow assay test strip,
at least one LED illumination source for illuminating a portion of a development area or a strip background region on the test strip and
at least one illumination sensor, for sensing illumination reflected or emitted from the portion of the development area on the test strip,
wherein the portion of the development area is one of a test line or a control line, on the test strip,
   wherein a current of electricity supplied to each LED illumination source is measured for detecting changes due to LED die temperature and changes in LED supply voltage during illumination of the lines on the test strip, and the changes used to calculate applied compensation.

In another aspect of embodiments, the invention provides an electronic reader for a lateral flow assay test strip, the electronic reader comprising,
an opening for receiving the lateral flow assay test strip, preferably a cassette containing the lateral flow assay test strip,
a PCB mounted on a carrier and including;
   at least one LED illumination source for illuminating a portion of a development area or a strip background region on the test strip, and
   at least one illumination sensor, for sensing illumination reflected or emitted from the illuminated portion of the development area on the test strip,
      wherein the illuminated portion of the development area is one of a test line or a control line on the test strip, and wherein each illumination source is paired with one illumination sensor.

Another aspect of embodiments provides an electronic lateral flow assay test reader for reading a lateral flow test strip, the electronic lateral flow assay test reader having a light guide comprising at least one window structure for framing a development area of the test strip, the development area comprising a test background region and at least one test result line, wherein the dimensions of the window structure are configured to maximise the proportion of the at least one test result line framed relative to the proportion of test background region framed.

Another embodiment provides a carrier of the reader which is adapted for engagement with a unitary housing of the reader and the carrier includes a window structure as disclosed herein.

In a preferred form, the test strip comprises masking features printed directly on its surface to isolate a result line from the test background region of the test strip. The test strip may then be inserted directly into the reader or into a cassette that is placed into the reader.

Alternatively, the test strip is inserted into a cassette, with the at least one window residing on the cassette.

The test result may be derived from the presence or absence of one or more test lines, determined by the presence or absence of a biomarker in the sample being tested, and/or a control line. Typically, the development area of the test strip would comprise at least one sample test line and at least one control line. The test strip may also comprise at least one strip background region.

Preferably the cassette comprises at least two windows for framing two or more portions of the development area of the test strip. The cassette may comprise two, three, four, five, six or seven windows, wherein each window frames a separate portion of the development area of the test strip. Equally, the cassette may comprise at least two windows for framing two or more respective development areas of the test strip, which provide for multiple test lines.

Preferably the cassette windows are aligned side by side along the length of the test strip.

In one embodiment, the cassette comprises one or more windows for separately framing one or more test result lines respectively, wherein the dimensions of each of the windows is configured to maximise the proportion of a test result line framed relative to the proportion of test background framed. In addition, the cassette may also comprise one or more windows for framing one or more control lines respectively, wherein the dimensions of each of the windows is configured to maximise the proportion of a control line framed relative to the proportion of test background framed. The cassette may also comprise at least one window for framing at least one strip background area of the test strip.

In a preferred embodiment, the dimensions of the cassette windows are configured such that the width of the window is equal to the width of the test or control line plus the tolerances of manufacture of one or a combination of the test strip and cassette. In this respect, the tolerances of manufacture may include the sum of the tolerance of the test line width, the tolerance of test line positioning on the test strip, the tolerance of test strip nesting in the cassette recess, and the tolerance of the window width.

Preferably, the electronic reader comprises at least one LED illumination source and at least one illumination sensor wherein each of the illumination source and illumination sensors are paired together.

Preferably, the carrier of the reader is adapted for engagement with a unitary housing of the reader. Typical lateral flow readers of the prior art include a housing comprising two or four parts that are fitted together rather than a unitary housing. Advantageously, the unitary housing reduces part inventory, complexity, assembly time, and provides mechanical protection for the PCB and carrier retained inside. In addition, as there is no seam in the unitary housing, the ingress of external ambient light into the reader is reduced ameliorating adverse effects on detection of the illumination sensors.

Preferably, the carrier provides a mount for the PCB and comprises windows. The carrier windows are configured to act as a light guide alone or in combination with the cassette windows when a cassette is inserted into the reader, such that only the light reflected or emitted from the test strip limited to the portion of the development area framed by the carrier and cassette windows and illuminated by the paired illumination LEDs is measured by the measurement sensor.

When the carrier windows are correctly aligned with the cassette windows, regions of the strip are able to be illuminated and are measurable by the paired illumination LED and measurement sensor. Essentially, the aligned carrier and cassette windows performs a masking function. The present inventors have found that separation or sharing of the masking function between the carrier windows and cassette windows allows the tolerance stack for positioning of the test line and control line within an area framed for measurement (the illuminated and measurable area) to be minimised. As a result, the present inventors have found that the test and control lines can be more accurately and repeatably positioned within separate and smaller windows when the windows are part of the cassette. Separate and smaller windows allowed the inventors to maximise the proportion of a test or control line framed relative to the proportion of background framed within the window, increasing the signal to noise ratio. In addition, by separating the light guide function into two parts, the masking features of the cassette windows can be placed closer to the test strip surface and the carrier windows (including the separator) can extended towards the PCB surface, to surround and separate the illumination LEDs from the measurement sensors. This in turn reduces the tolerance stack. The cassette windows may prevent regions of the strip such as the edges from being measured. In this regard, the cassette window is arranged to mask the sides of the test strip so as to minimise exposure of the amount of the strip that contains non-uniform non-specific binding.

Another advantage of separating the light guide function between the carrier and the cassette is that the carrier windows (including the separator) can extend towards the PCB surface to surround and separate the illumination LEDs from the measurement sensors, whilst allowing for other masking features to be placed in close proximity to the lateral flow strip as part of the cassette windows. The carrier windows act to reduce the light from an illumination LED reaching neighbouring regions on the test strip and reflecting back to the sensor of a LED/sensor pair. In addition, the carrier windows are designed to minimise the illumination and measurement of reflected light from the cassette windows and cassette surface, reducing interfering signal noise. A preferred embodiment of the present invention locates an outer frame for the window close to the strip (the cassette window) and locates a secondary frame close to the LED and sensor (carrier window).

In one embodiment, each carrier window comprises a LED window and a sensor window separated by a barrier (or separator) which prevents the light from the illumination LED from reaching the measurement sensor directly, allowing for the measurement of the reflected or emitted light from the test strip.

In still yet a further aspect of embodiments described herein there is provided an electronic reader for a lateral flow assay test strip, the electronic reader comprising,
an opening for receiving a lateral flow assay test strip, preferably a cassette containing the lateral flow assay test strip,
at least one LED illumination source for illuminating a portion of a development area on the test strip, and,
at least one illumination sensor, for sensing illumination reflected or emitted from the illuminated portion of the development area on the test strip,
wherein the illuminated portion of the development area is one of a test line, a control line, or a strip background region on the test strip,
wherein the cassette is removably retained within the reader by a snap fit mechanism

The elements of the snap fit mechanism may reside upon or within the cassette and/or the reader and their assistance with alignment of the cassette within the reader contributes to consistent and correct measurements.

In yet another aspect of embodiments described herein there is provided an electronic reader for a lateral flow assay, the electronic reader comprising,
an opening for receiving a lateral flow assay test strip, preferably a cassette containing the lateral flow assay test strip,
at least one LED illumination source for illuminating a portion of a development area on the test strip, and
at least one illumination sensor, for sensing illumination reflected or emitted from the portion of the development area on the test strip,
wherein the portion of the development area is one of a test line or a control line,
wherein the reader further comprises input/output (IO) pins where each respective IO pin is operatively associated with two or more LEDs of the reader.

The electronic architecture of embodiments of the present invention allows the use of a greater number of measurement positions and user feedback LEDs than are usually provided with low cost microcontrollers of the prior art. Typically, in prior art each IO pin controls a single LED. A preferred embodiment of the present invention instead uses a combination of charlieplexing and multiplexing to control multiple LEDs (e.g. twelve, six user feedback LEDs and six illumination LEDs) from five digital IO pins. While this configuration has the apparent drawback of only a single LED being powered at once, it has the advantage of predictable and low current draw from the battery. Herein below, there is description of how rapid switching of the user feedback LEDs can be used to give the appearance of multiple LEDs being on simultaneously.

The reader comprises a user feedback system to communicate with the user. The user feedback system can be used to communicate the state of the reader to the user (such as cassette inserted, test in progress or test complete), communicate the test result and/or the validity of the test. Preferably, the user feedback system comprises a plurality of user feedback LEDs, wherein the LEDs are used as indicators to communicate to the user. Alternatively, the user feedback system may comprise an LCD screen for displaying the result and/or communicating the state of the reader with the user.

Optionally, the user feedback system comprises connectivity elements, such that the reader can communicate to an external device. The external device may be a smartphone or computer which can be used to communicate the state of the reader and/or communicate the test results. The external device may also process the information communicated by the reader and interpret the data in order to communicate the test result. Connectivity elements may include wireless connectivity such as WIFI or Bluetooth.

Furthermore, incorporating multiple LEDs into the lateral flow assay device allows the inclusion of other functionality such as a cassette presence/absence detection feature. The following feature can be implemented using the LEDs and sensors already provided for user feedback and test measurement. When there is no cassette inserted, the light from one of the user feedback LEDs reaches the measurement region and can be detected by one or more of the measurement sensors. When the cassette is inserted, the user feedback LED light is blocked by the cassette and does not reach the one or more measurement sensors. This way the user experience is improved by reducing the number of required interactions prior to performing a test. This user feedback is implemented in software without any additional components.

In another embodiment, the reader comprises a normally open reset switch, wherein the switch is located inside the reader and is activated when a cassette is inserted or removed. This allows the reader to be in a low power state until a user interacts with it by inserting or removing a cassette, decreasing the power consumption requirement. This increases the shelf life of the reader and permits a lower capacity, less expensive battery to be used.

A combination of the reader reset switch and the cassette detection features can be used in software to determine what the user intends to do. For example, if the reset switch is toggled and a cassette is detected, it is likely that the user has inserted a cassette and intends to start a test. The alternative scenario is if the reset switch is toggled and there is no cassette detected, then it is likely that the user has just removed a cassette, the powered-on reader can now continue to perform functions such as displaying the result of the previously completed test or maintaining communication with an external device.

In a further embodiment, an aforementioned embodiment of the lateral flow assay electronic reader of the present invention is combined with the aforementioned cassette.

Preferably the snap fit mechanism comprises biasing springs associated with the reader carrier and snap fingers on the cassette which work together to ensure that the cassette windows substantially align with the carrier windows. Preferably, the result lines of the test strip are centred in respect to the substantially aligned carrier and cassette windows to ensure that illumination and measurement of the signal at the test and/or control line is optimised. The biasing springs associated with the reader carrier and snap fingers on the cassette work together wherein the biasing means pushes the cassette out towards the opening and snap fingers on the cassette stop the cassette from leaving the reader. The retaining or retention mechanism holds the cassette in place within the reader and aligns cassette and reader features. This ensures correct and consistent readings.

The cassette is removably retained within the reader, such that the snap fingers of the cassette can be depressed and the biasing means assists in releasing the cassette from the reader opening.

When the cassette is positioned optimally in the reader, the cassette windows may align with the carrier windows that frame the illumination LEDs and measurement sensors.

The present invention further provides a system comprising the cassette and the electronic reader of the present invention.

The present invention also provides a method of assessing result lines of a lateral flow assay test strip comprising the steps of;
(i) inserting the cassette containing the assay test strip into a reader according to the present invention; and
(ii) applying the sample that needs to be measured onto the cassette; and
(iii) initiating the illumination source of the reader and detecting illumination reflected or emitted from the assay test strip.

A multiuse reader which can be used to read more than one cassette is also disclosed. In one embodiment, the multiuse reader is a self-contained unit including a reader door that prevents contaminants from entering a cavity of the multiuse reader when a cassette is not installed in the multiuse reader. Once a cassette is inserted through the opening, the reader door pivots on a hinge. Alignment features such as location pins, alignment pins, retaining clips and other features are used to align and secure the cassette within the reader. The alignment features can be present on or within the cassette, the reader or a combination of both the reader and the cassette.

In another embodiment, a multiuse reader clips onto a cassette via clips of the reader surrounding the cassette or sides of the multiuse reader being received within corresponding recesses on the side of the cassette.

In another embodiment, a multiuse reader slides onto the cassette via a set of rails present on the cassette and/or within the reader itself.

Other aspects and preferred forms are disclosed in the specification and/or defined in the appended claims, forming a part of the description of the invention.

In essence, embodiments of the present invention stem from the realisation that the level of sensitivity of detection of lines in the development area of an assay test strip can be improved by one or more electronic, mechanical and software features, which work adequately in isolation but provide significantly better results when used in various combinations.

Advantages provided by the present invention in comparison to the prior art comprise the following:
improvement in reader performance, avoiding the need for adjustment of test strip chemistry,
improvement in sensitivity,
reduction of background noise with increased resolution of measurable test results,
improved alignment and positioning of result lines relative to electronic reader measurement area;
the cassettes are disposable, low cost to manufacture and assemble,
the readers are ultimately disposable, for single use or low volume based testing, and are low cost to manufacture and assemble,
the reader is of simple configuration yet provides reduced energy consumption when not in use,
Reduction in signal from areas not directly associated with the region being measured leads to improved sensitivity
Improved alignment and positioning of result lines leads to improved accuracy.
Improved isolation between measurement regions allows simple extension to support additional result lines.
Improved use of processor I/O resources allows simple and low-cost expansion to support additional result lines.
A low-cost technique for driving and correcting LED performance.

Further scope of applicability of embodiments of the present invention will become apparent from the detailed description given hereinafter. However, it should be understood that the detailed description and specific examples, while indicating preferred embodiments of the invention, are given by way of illustration only, since various changes and modifications within the spirit and scope of the disclosure herein will become apparent to those skilled in the art from this detailed description.

### BRIEF DESCRIPTION OF THE DRAWINGS

Further disclosure, objects, advantages and aspects of preferred and other embodiments of the present invention may be better understood by those skilled in the relevant art by reference to the following description of embodiments taken in conjunction with the accompanying drawings, which are given by way of illustration only, and thus are not limitative of the disclosure herein, and in which:

FIG. 1 illustrates a typical lateral flow test strip of the prior art;

FIG. 2A and FIG. 2B are exploded and assembled illustrations of a preferred embodiment of the present invention, respectively;

FIG. 3 illustrates an exemplary cassette containing an assay test strip in accordance with an embodiment of the present invention where FIG. 3A shows a cassette comprising a plurality of windows and FIG. 3B shows a single cassette window with masking features directly on the test strip;

FIG. 4 illustrates a cassette window configured to a test result line in accordance with an embodiment of the present invention;

FIG. 5 illustrates the framing of test result lines of a test strip by cassette windows in accordance with embodiments of the present invention, where FIG. 5A and FIG. 5B show acceptably framed test result lines and FIG. 5C shows an unacceptable framing of a test result line;

FIG. 6 is a side sectional view of a PCB mounted on a carrier in accordance with an embodiment of the present invention;

FIG. 7A is a bottom sectional view of a cassette showing a PCB mounted on a carrier in accordance with an embodiment of the present invention, FIG. 7B is a detail view of the measurement area of the carrier;

FIG. 8A is a top view of a cassette inserted into an opening in a carrier, and FIG. 8B is an in-section view showing the cassette of FIG. 8A with test strip nested therein and inserted into the reader carrier in accordance with an embodiment of the present invention;

FIG. 9 is a plot of measured attenuation against test line intensity comparing the performance of black and white cassettes with the reader according to an embodiment of the present invention;

FIG. 10 is a section view illustrating operation of a cassette in association with a reader of according to an embodiment of the present invention where FIG. 10A shows an open reset switch, FIG. 10C shows a closed reset switch and, FIG. 10B shows a reset switch re-opened on removal of the cassette from the reader;

FIG. 11 is a schematic electronic circuit diagram illustrating a basic arrangement of LEDs according to preferred embodiments of the present invention;

FIG. 12 is a table showing charlieplexing and multiplexing control, respectively, for a varying number of loads as a function of the number of available I/O pins utilised in a reader according to a preferred embodiment of the present invention;

FIG. 13 shows another embodiment of a reader of the present invention for detection of the presence of a cassette inserted in a carrier (FIG. 13A) and for detection of the absence of a cassette inserted in a carrier (FIG. 13B).

FIG. 14A is a cross sectional view of a cassette and strip inserted within a multi-use reader carrier in accordance with an embodiment of the present invention, illustrating that the light guide function is separated between the carrier and the cassette. FIG. 14B is a detailed view of the cross section of FIG. 14A showing illumination paths associated with an illumination LED and measurement sensor pair for a portion of the development area of the test strip. FIG. 14C illustrates the respective areas of the test strip that are illuminated and measurable in accordance with the embodiment of FIG. 14A.

FIG. 15A and FIG. 15B are exploded and assembled illustrations, respectively, of a single use version of a preferred embodiment of the present invention where the test strip is contained in the reader without a cassette or carrier as such, and in which the top and bottom housing may be considered to serve the function of a carrier.

FIG. 16A and FIG. 16B are section views showing an overlay of the LED and sensor locations on top of the carrier and cassette assembly. FIG. 16C is a detailed view of the cassette inside the carrier and FIG. 16D is a detailed view of the carrier only.

FIG. 17A and FIG. 17B are 3D section views illustrating a cassette fully inserted into a carrier and the reset switch on the PCB.

FIG. 18A and FIG. 18B are side section views of a cassette inserted in a carrier showing the alignment of the cassette windows and the carrier windows.

FIGS. 19A, 19B, 19C and 19D are different views of a multiuse reader for use with a cassette assembly, with FIGS. 19B, 19C, and 19D showing sectional views of the multiuse reader.

FIG. 20A and FIG. 20B show a multiuse reader and a close-up view of a reader door in the closed and open positions respectively.

FIG. 21 shows a sectional view of a multiuse reader with an inserted cassette.

FIG. 22A and FIG. 22B show a sectional view of the cassette in a multiuse reader.

FIG. 23A and FIG. 23B show closeup views of a printed circuit board assembly.

FIG. 24A and FIG. 24B are schematic electronic circuit diagrams illustrating a simplified architecture to drive a multiplexed LCD arrangement.

FIG. 25 shows a top down sectional view of a multiuse reader with an inserted cassette with the top removed.

FIG. 26 shows a sectional view of the cassette within a multiuse reader.

FIG. 27A and FIG. 27B are sectional views of a multiuse reader receiving a cassette and being aligned within a multiuse reader.

FIG. 28A and FIG. 28B show a blood collection unit blocker on a multiuse reader.

FIG. 29A to FIG. 29D show views of a cassette with a reader which is slid on.

FIG. 30A and FIG. 30B show views of a clip-on multiuse reader attached to a cassette.

FIG. 31A shows a sectional view of a clip-on multiuse reader attached to a cassette.

FIG. 31B shows a closeup view of a locating pin of the clip-on multiuse reader.

FIG. 32A shows a clip-on multiuse reader.

FIG. 32B shows a partial sectional view of a clip-on multiuse reader.

FIG. 33 shows an exploded view of a clip-on multiuse reader without a reader cover.

### DETAILED DESCRIPTION

The following is a component list for figure reference numerals as depicted in the accompanying drawings:

Biological sample 1
Sample pad 2
Direction of flow 3
Conjugate pad 4
Test result line 5
Background region 6 (which may include both strip background and
test background) 6
Control line 7
Development area 8
Nitrocellulose membrane 9
Waste pad 10
Backing card 11
Cassette top 12
Test strip 13
Cassette bottom 14
Cassette assembly 15
PCB 16 (Printed Circuit Board)
Carrier 17
Reader opening 18
Battery 19
Housing 20
User feedback LEDs 21
Sample port 22
Snap fingers 23
Cassette window structure 24
Direct masking 25
Viewing area 26
Activation recess 27
Width of test result line 28
Combined tolerance for test result line 29
Width of window 30
Height of window 31
Height of test strip 32
Region of non-uniform non-specific binding 33
Leaf springs 34 (vertical biasing means)
Lateral biasing means 35
Reset spring clip 36
Measurement sensors 37
Illumination LEDs 38
Measurement area 39
Carrier windows 40
Switch open 41
Switch closed 42
Illumination and sensor separator 43
Adjacent sensor separator 44
Measurement shadow 45
Area framed for measurement 46
Illumination shadow 47
Housing top 48
Housing bottom 49
Light guide 50
Multiuse reader 51
Reader housing 52
Reader housing top 53
Reader housing bottom 54
Output user interface 55
Reader door 56
Reader dock 57
Door pin 58
Reader door socket 59
Spring clip 60
Alignment recess of the door 61
Locating boss 62
Reader cavity 63
End posts 64
Spring return feature 65
U-shaped recess 66
Lip interface 67
Door lip 68
Door receiving section 69
Alignment section 70
Cassette 71
Retention Clips 72
Cassette detection switch 73
Raised surface of cassette 74
Cassette bump 75
Channel on cassette 76
Cassette top 77
Printed circuit board assembly (PCBA) 78
Battery terminal 79
Optics components 80
Thin rib 81
Alignment pin 82
Blood collection unit of cassette 83
Blood collection tube of cassette 84
Sample port of cassette 85
Buffer delivery button 86
Alignment boss 87
Light guide 88
Ramps of cassette 89
Blood collection unit blocker (BCU) of reader 90
Rails/rib on cassette 91
Slide-on multiuse reader 92
Shroud of slide-on multiuse reader 93
End stop 94
Cassette bottom 95
Sliding feature of reader 96
Clip-on multiuse reader 97
Clip-on arms 98
Cassette recess 99
Shoulder 100
Alternate clip-on reader 101
Cassette with recess 102
Cassette recess 103
Reader bottom 104
Rounded face of clips 105

FIG. 1 illustrates a typical lateral flow test strip 13 of the prior art but which may also find use in the present invention. Lateral flow assays are immunoassay based diagnostic tests and are often configured in the form of a test strip 13 or card to which various testing components are attached. In essence, they rely on capillary flow of liquid through a membrane containing a capture reagent.

The illustration of FIG. 1 depicts droplets of a biological sample 1 being dropped in the direction of the arrow 1 onto a treated sample pad 2 on a test strip 13 of polymeric backing card 11. The adjacent pad (conjugate pad) 4 is soaked with a labelled detector reagent (conjugate), such as a gold colloid or fluorescent labelled microparticles conjugated to a detector antibody. The conjugate is reconstituted and binds any analyte in the sample if present. The conjugate and sample flows in the direction of the arrow 3 through the nitrocellulose membrane 9, passing the capture antibodies which may eventually develop into the test line 5 and control line 7, further indicated with a “T” and a “C”, respectively, as shown, as well as background regions 6 without capture antibodies, which may include strip background and test background and, ultimately ending at the waste pad 10. After a predetermined amount of time, the test is deemed completed and the development area 8 is inspected to determine the test result.

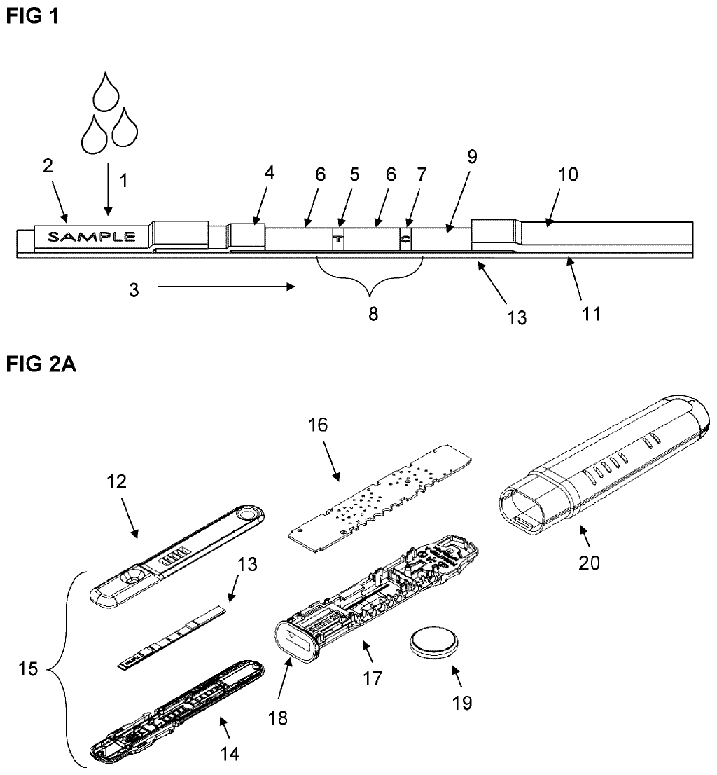

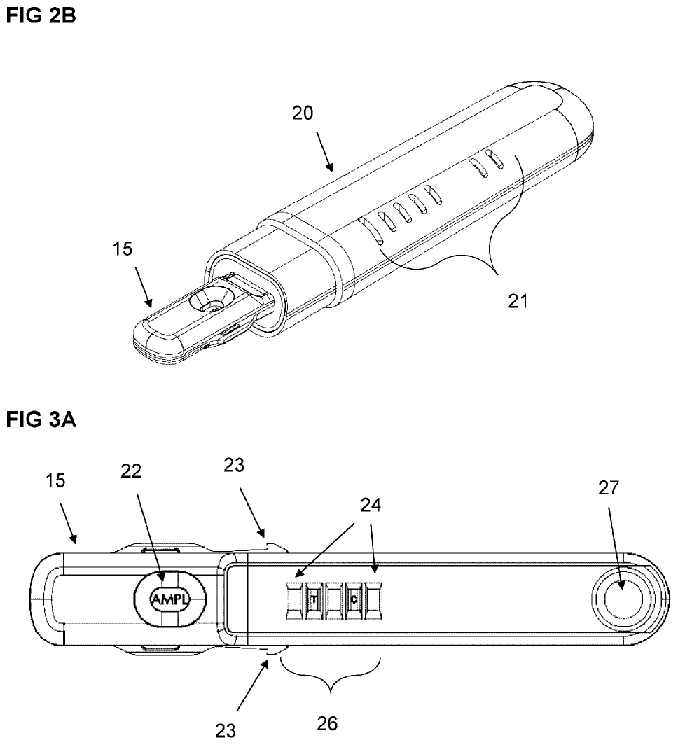

The illustration of FIG. 2A and FIG. 2B depict the lateral flow assay electronic reader of a preferred embodiment of the present invention comprising a PCB 16 mounted on a carrier 17, a battery 19, encased in a unitary housing 20. The carrier 17 contains an opening 18 which accepts a cassette assembly 15 where the cassette assembly 15 comprises a cassette top 12, cassette bottom 14 and lateral flow test strip 13. The PCB 16 holds user feedback LEDs that are visible through holes or apertures 21 in the carrier, as shown in FIG. 2B.

The unitary housing 20 reduces part inventory, complexity, assembly time, and provides mechanical protection for the PCB 16 and carrier 17 retained inside. In addition, as there is no seam in the unitary housing 20 the ingress of external ambient light into the reader is reduced. Another advantage of a unitary housing 20 is the lack of side seams also means the ingress of external fluid from the environment, such as cleaning fluid, is reduced and the internal electronic components are protected.

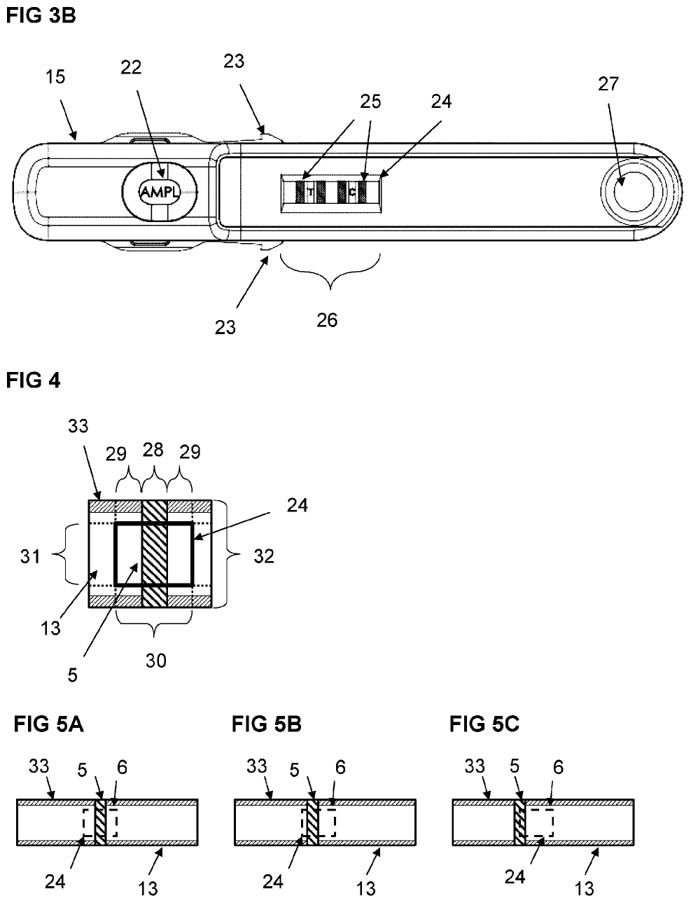

FIG. 3 illustrates a preferred embodiment of the cassette 15 containing an assay test strip 13. FIG. 3A depicts features of the cassette assembly 15, comprising a sample port 22, snap fingers 23, a viewing area 26 comprising a cassette window structure 24 having a plurality of windows in this instance for isolating or masking portions of the development area 8 of the test strip 13, where the dimensions of the window(s) are configured to maximise the proportion of test result lines 5 framed relative to the proportion of test background framed. The cassette assembly 15 also includes a reset activation recess 27. Again, it is noted that the plurality of windows of the cassette window structure 24 in FIG. 3A serves to mask the test strip 13. FIG. 3B illustrates an alternate arrangement wherein the viewing area 26 includes a cassette window structure 24 which is one large window and the portions of the development area 8 are framed with masking features 25 integrated on the test strip 13, such that the masking is configured to maximise the proportion of result lines relative to the proportion of test background.

For a singleplex assay with one test line 5 and one control line 7, at least three windows are required, one window for the test line, one window for the control line 7 and at least one window for the strip background. Preferably, four windows with two windows for strip background measurements improve test sensitivity. In this preferred configuration, the first and third windows are each for a strip background calibration measurement the second window is for the test line and the fourth window is for the control line 7. Optionally, the background calibration measurement can be reduced to a single strip background calibration area in the first window. For multiplex assays with two or more test lines, the second and third window each frame one test line, with further additional windows provided for each additional test line over two test lines. For a cassette 15 with five windows 24 as depicted in FIG. 3A, the maximum number of test lines 5 would be three, where there has to be at least one strip background region 6 and there could be three test lines 5 and one control line 7.

FIG. 4 illustrates how the cassette is configured such that a test result line 5 of the test strip 13 is positioned within a cassette window structure 24. The combined tolerance 29 of the cassette recess that nests the test strip and test strip 13 itself (including tolerance of the result line width, tolerance of position of the result line on the test strip, tolerance of position in the cassette, tolerance of window size and safety factor) are sufficient to ensure that the full width 28 (parallel to the direction of flow 3) of each result line is positioned within the width of the cassette window 30. The height of the window 31 is configured to the test strip width 32, excluding the lateral edges 33 where non-uniform non-specific binding is expected to occur.

In the embodiment of FIG. 4, the dimensions of the cassette windows 24 are configured such that the width of the window is equal to the width of the test or control line plus the tolerances of manufacture of one or a combination of the test strip 13 and cassette 15. In this respect, the tolerances of manufacture may include the sum of the tolerance of the test line width 28, the tolerance of test line positioning on the test strip 13, the tolerance of test strip nesting in the cassette recess, and the tolerance of the window width 30. For example, a 1.5 mm wide test line would be framed by a window at least 1.5 mm wide, wherein the width of the window is 1.5 mm plus the tolerances of manufacture. In this example, in combination with controlled manufacturing processes, the window width may be around 2.5 mm to allow for the realistically expected manufacturing tolerances. In use, the cassette is removably inserted in an electronic reader, which comprises an illumination source for illuminating the test result lines 5 and test background regions 6 on a lateral flow test strip 13, and measurement sensors 37 for detecting light reflected or emitted from the test lines 5.

The cassette 15 is configured such that each result line of the test strip 13 is positioned or aligned for inspection within a separate cassette window 24. The tolerance of the cassette recess which nests the test strip and test strip itself should be sufficient to ensure that the full width (parallel to the direction of flow 3) of each result line is positioned within a cassette window 24. Because these tolerances are known and tightly controlled the windows can be sized as small as possible while ensuring the full width of each of the result lines is positioned within a separate window. This ensures that the signal measured from the result line is maximised and signal from the test background is minimised. The cassette and test strip tolerances should be accommodated to ensure that the entirety of the line remains in the window and visible to the entirety of the LED and photodiode active surface areas when the cassette and strip tolerances are all at their worst-case extremity. If the cassette window is misaligned with respect to the carrier window 40 along the long axis of the cassette, it has little impact on the signal since there is no additional obscuration of the line due to alignment error (as the carrier window is designed to be sufficiently larger than the cassette window that it allows for this alignment error and the whole of the cassette window remains “visible”) The alignment position error may contribute a cosine error due to small angular changes, as does the line position within the cassette window.

The height of the cassette windows 31 (perpendicular to the flow) are smaller than the full width of the test strip to reduce the interference from edge artefacts. The edges of a lateral flow test strip 13 tend to have non-uniform and or non-specific binding of analytes and/or antibodies producing resultant artefacts, which adds additional noise to the overall signal derived from test and control lines.

The cassette window height 31 is sized such that there is a balance between maximising the amount of test strip exposed for measurement and excluding the interference from the above noted edge artefacts. Preferably, the cassette window height is such that the height of window is less than or equal to the test strip width (perpendicular to the flow) minus manufacturing tolerances. The manufacturing tolerances for window height includes, the test strip width, the tolerance of test strip nesting in the cassette recess, and the tolerance of the cassette window.

In a preferred embodiment, around 0.35-0.40 mm of the test strip edging is covered on each side of the test strip by the cassette housing on each side of the cassette window, wherein the cassette window is centred in relation to the test strip when nested in the recess of the cassette. For example, the cassette window height is around 3.25 mm +/−0.05 mm high for a 4 mm wide test strip. For a 6 mm wide test strip the cassette window height is around 5.25 mm +/−0.05 mm, and for a 2 mm wide test strip it would be around 1.25 mm +/−0.05 mm.

FIG. 5 illustrates how the cassette window structure 24 is intended to frame the result line 5 of the test strip. FIG. 5A illustrates a result line ideally centred in the cassette window 24, FIG. 5B illustrates a result line 5 where the full width is positioned within the cassette window 24, and FIG. 5C illustrates a result line 5 that is overlapping the cassette window 24 and partially obscured by the cassette housing. The proportion of result line 5 and test background region 6 positioned within the window 24 is equal in FIG. 5A and FIG. 5B but not in FIG. 5C.

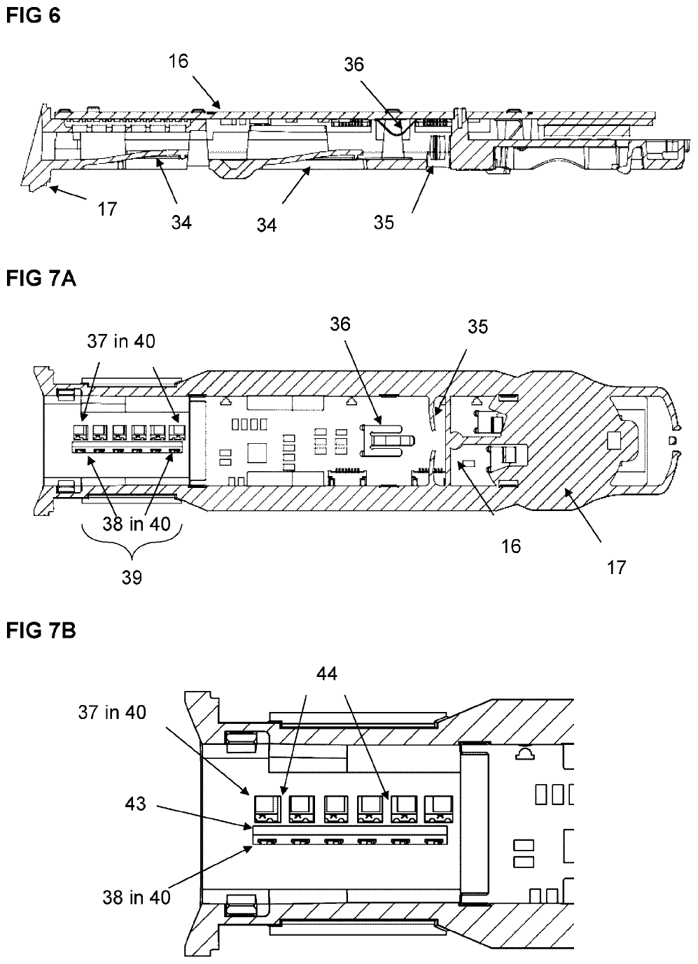

FIG. 6 illustrates a side sectional view of the PCB 16 mounted on the carrier 17. FIG. 7A illustrates a sectional view of the PCB 16 mounted on the carrier 17 as viewed from the bottom, FIG. 7B is a detail view of the carrier windows 40 showing the light and sensor separator feature 43, parallel to the direction of flow 3 on the test strip, which prevents the light from the illumination LED 38 from reaching the measurement sensor 37 directly. This arrangement allows for the measurement of the reflected or emitted light from the test strip 13. The adjacent sensor separators 44 perpendicular to the direction of flow 3 frames the window around the sensor and prevents light reflected or emitted from adjacent windows from reaching the measurement sensors. In one embodiment as shown in FIG. 7B, the active areas of measurement sensor 37 and LED 38 pairs are offset so as to fit a plurality of sensors 37 (in this example six sensors) within the standard lateral flow strip dimensions to maximise the number of areas that can be separately measured on a lateral flow test strip. In another embodiment, the centres of the active areas of the light source 38 and sensor 37 pairs are uniformly aligned and each pair is centred within the aligned carrier and cassette windows.

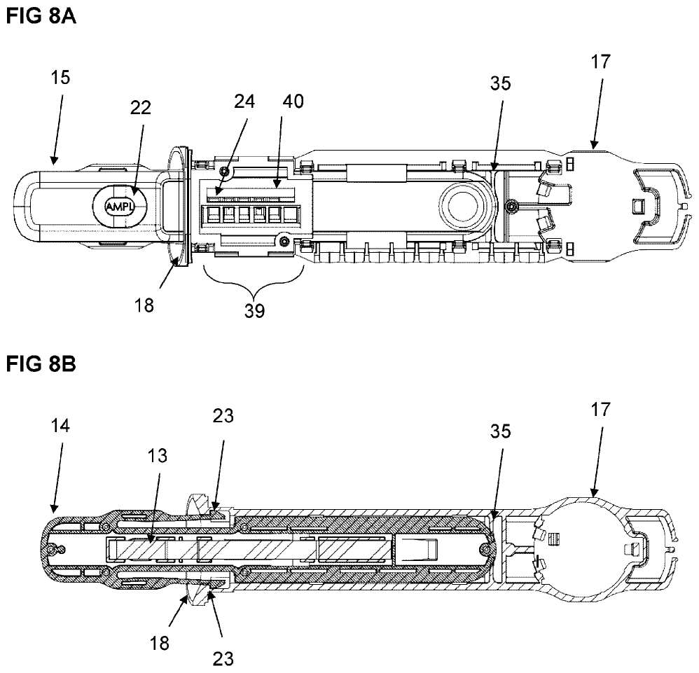

FIG. 8A illustrates a view of the cassette 15 inserted into the opening 18 in the carrier 17, FIG. 8B is a sectional view of the cassette with test strip inserted into the reader carrier.

In a preferred embodiment the cassette is removably retained within the reader by a snap fit mechanism. The elements of the snap fit mechanism may reside upon or within the cassette and/or the reader and their assistance with alignment of the cassette within the reader contributes to consistent and correct measurements.

As would be appreciated by the person skilled in the art, any suitable snap-fit mechanism may be employed and may comprise annular, cantilever or torsional snap-fit arrangements. Preferably a cantilever snap-fit mechanism is employed. The snap fit mechanism in a particularly preferred embodiment comprises a snap fit retaining mechanism and lateral biasing means to retain and align the cassette within the reader. The lateral biasing means may comprise spring elements which may be separate or integral spring features such as leaf or coil springs, or alternatively the inherent structural compliance of the reader and or cassette components may be employed, particularly as these components are constructed of polymeric materials. In a preferred embodiment, the snap fit mechanism comprises lateral biasing means on the carrier and snap fingers on the cassette (or alternatively in a mechanical inversion, lateral biasing means on the cassette and snap fingers in the reader) work together to ensure that the cassette is ultimately positioned consistently and correctly in the reader.

Preferably, the lateral biasing means and the snap fingers work together such that the lateral biasing means push the cassette out towards the opening of the reader and the snap fingers act as a retaining mechanism to retain the cassette within the reader. Together the elements of the snap fit mechanism hold the cassette in a reading position within the reader. When the cassette is nested optimally in the reader, the cassette windows align with the carrier windows that frame the illumination LEDs and measurement sensors. Misalignment of the cassette windows and carrier windows would impact on the signal measured as misaligned windows would obscure the result lines and ultimately reduce measurement performance. Preferably, the snap fit mechanism aligns the cassette and reader carrier windows such that the position of each test result line 5 is centred in the aligned respective windows. This alignment of the cassette within the reader contributes to consistent and correct measurements.

Other retaining mechanisms such as retention clips on the reader which engage features on the cassette can be used to align and retain the cassette within the reader. Additional retaining features such as alignment pins and associated holes or bosses can also be used to retain the cassette within the reader and secure alignment within the reader.

In a particularly preferred embodiment, the reader also comprises vertical biasing means for positioning the cassette vertically towards the measurement area. Preferably, the vertical biasing means comprise one or more leaf springs that urge the cassette towards the electronic components or the reader used for measuring. This contributes to maintaining a consistent distance between the assay test strip and the electronic components used for measuring, and hence consistent measurement. Due to light scattering, not all of the light emitted by the illumination LED reaches the test line, and not all the light reflected or emitted by the test line is detected by the measurement sensor. A consistent distance between the assay test strip and the measurement region ensures that the same proportion of light is detected by the measurement sensors.

Preferably, the vertical biasing means comprises two leaf springs that urge the cassette towards the electronic components used for measuring, wherein the first leaf spring urges the cassette towards the electronic components for measuring, such that the cassette windows and carrier windows are in contact and wherein the second leaf spring maintains the cassette parallel with the PCB.

Preferably the cassette 15 is removably retained within the reader by a snap fit mechanism comprising snap fingers 23 and a biasing means 35. The snap fingers on the cassette 23 and the biasing means 35 on the reader carrier 17 ensure that the cassette windows are aligned correctly in relation to the measurement area 39. The measurement area 39 comprises carrier windows 40, which are divided by a barrier 43 to act as a light guide for the measuring system comprising illumination LEDs 38 for illuminating the result lines 5 and 7, and test background regions 6 of the test strip and electronic measurement sensors 37 for sensing light reflected or emitted from the test strip. Preferably one LED is paired with one sensor to illuminate and measure signal at one portion of the development area 8, such as the test line 5, control line 7 or strip background region 6. Additional LED-sensor pairs are used to measure another portion of the development area 8 of the test strip. Preferably the windows 24 in the viewing area 26 of the cassette are centred with the windows 40 in the measurement area 39 of the carrier.

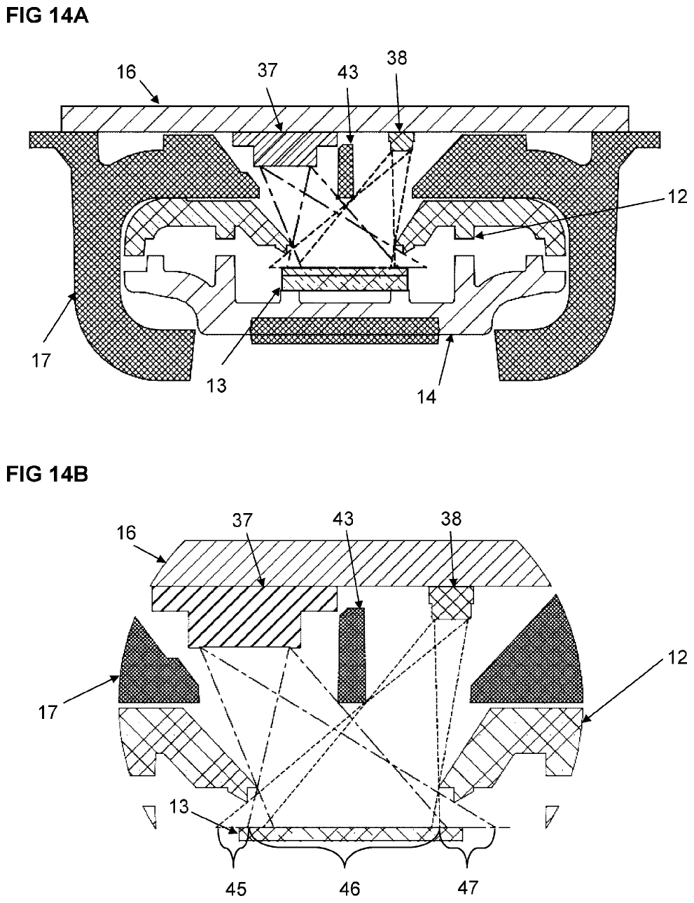

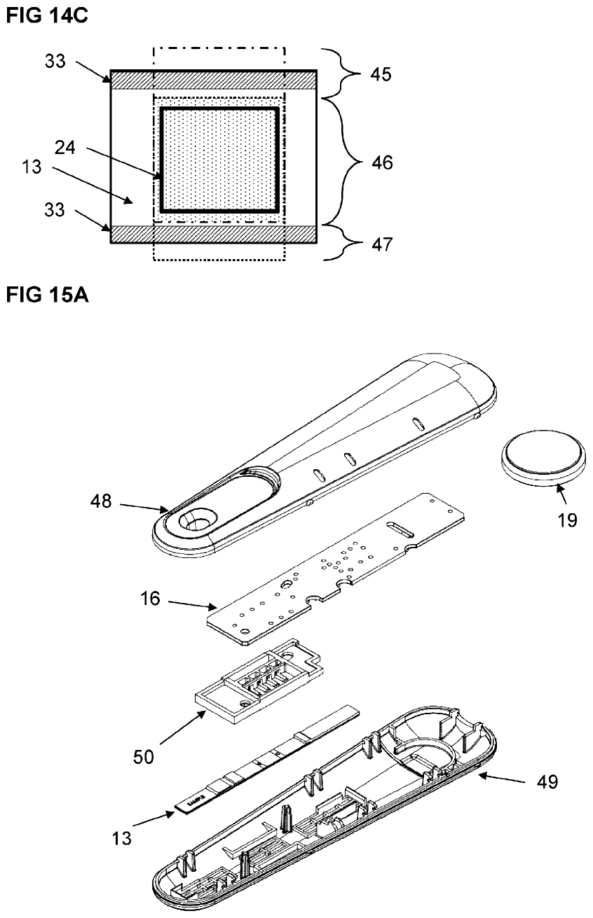

In a particularly preferred embodiment the biasing means are leaf springs 34 that urge the cassette 15 towards the electronic components used for measuring. FIG. 6 is a sectional view illustration of a particularly preferred embodiment of a carrier 17 of an electronic reader according to the present invention having two leaf springs 34 that help align the cassette vertically to the reader. In this arrangement, one leaf spring pushes the cassette so that the carrier 17 and cassette 15 are in contact and the second leaf spring maintains the cassette parallel to the PCB 16. The leaf springs 34 assist in maintaining a consistent distance between the assay test strip in the inserted cassette and the electronic components of the reader used for measuring, and hence reduces measurement variables by maintaining a consistent measurement depth. The distance between the test strip and measurement components are optimised in order to position the overlap of illumination area and measurable area on the area framed for measurement. Depicted in FIG. 14A and FIG. 14B is a detailed view of the cross section of a cassette 15 in the carrier 17 showing illumination paths associated with an illumination LED 38 and measurement sensor 37 pair for a portion of the development area 8 of the test strip 13. With respect to the test strip to LED/sensor distance, the inverse-square law operates. However, the present inventors have found that because of the limited “field of view” of the LED 38 and sensor 37 and the associated geometry, there is a point beyond which further reduction in separation distance actually reduces signal rather than increasing signal. The present inventors found that the area framed for measurement 46 was optimal at a test strip to PCB distance of between about 2 mm and 5 mm. Preferably, strip to PCB distance of about 3 mm to 4.5 mm. More preferably, strip to PCB distance of about 4.1 to 4.5 mm.

The present inventors found that by separating of the light guide function between the carrier 17 and cassette 15 that they could optimise the light guide function. In this arrangement, the carrier windows 40 (including the separator 43) can extend towards the PCB 16 surface to surround and separate the illumination LEDs 38 from the measurement sensors 37, whilst allowing for other masking features to be placed in close proximity to the lateral flow strip 13 as a plurality of cassette windows 24. Allowing the inventors to minimise the distance between the test strip 13 and the cassette window 24. For functionality reasons, the distance between the top surface of the test strip 13 and the bottom surface of the cassette windows includes an air “gap” such that the cassette windows 24 do not directly contact the test strip 13 surface as such contact may interfere with flow of sample solution along the test strip 13. Since this distance represents a “gap” it leads to the creation of shadows which act to restrict or provide a limitation to the illuminated portion of the test strip 13. These shadows are dependent on both the distance between the strip and the cassette window and also distance of the strip to the LED/sensor pair. The shadows are caused by the interaction of light paths, the carrier window 40, the cassette window 24 and their relative locations, which is evident with reference to FIG. 14B.

The cassette is configured such that each result line of the test strip is positioned within a separate cassette window, and at least one strip background region 6 is framed by a separate cassette window 24. The tolerance of the cassette window 24 and test strip in terms of manufacturing and assembly are sufficient to ensure that the full width (parallel to the flow) of each result line is positioned within a window 24. Because these tolerances are known and tightly controlled the windows 24 can be sized as small as possible while ensuring the full width of the each of the result lines is positioned within a separate window 24. This ensures that the signal measured from the line is maximised and signal from the test background is minimised as per FIG. 5.

Cassettes of the prior art are typically white, or a light colour such as pink, light blue or light green to provide visual contrast to the darker test lines. However, counter intuitively to this, it has been recognised that the use of minimally reflective cassette colour such as black improves the reader contrast. A minimally reflective cassette means that less light is reflected off the cassette and into the measurement sensors. The term ‘minimally reflective’ is intended to include any combination of surface and colour that is non-reflective or absorbs wavelength of the illumination source in the electronic reader. This helps reduce the reflected light from the ambient environment and prevents the reflected light from straying into neighbouring measurement areas. Furthermore, it contributes to maximising detection of reflection from the test result line 5 and reducing background region signal noise.

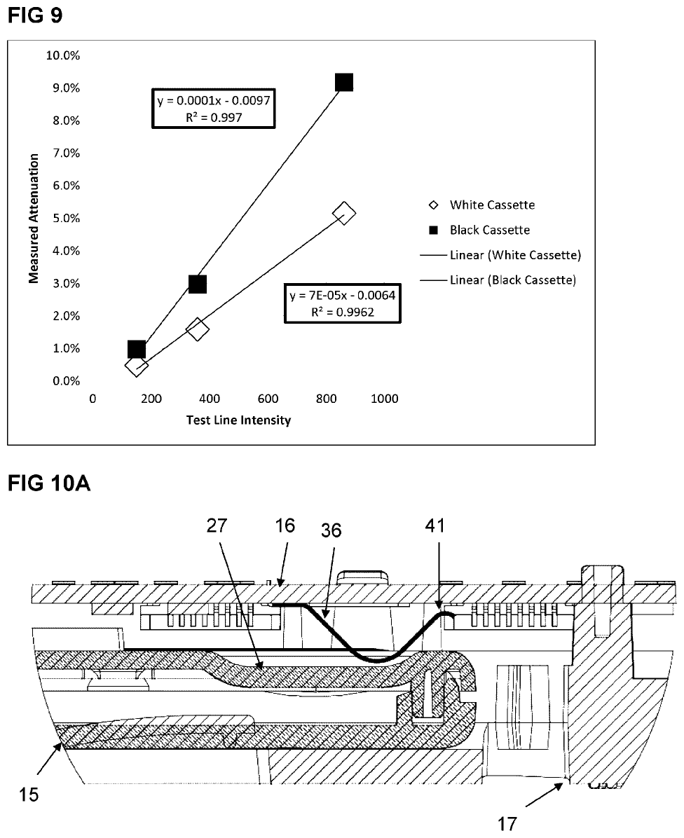

FIG. 9 is a plot of measured attenuation against line intensity comparing the performance of black and white cassettes with a reader of a preferred embodiment of the present invention. Black and white cassettes were tested with three lateral flow test strips with varying line intensities. Each test strip was placed in 5 white cassettes and 5 black cassettes and measured in the reader of the present invention. On average the test strip in the black cassettes had 75% higher attenuation than the same test strips in the white cassettes.

The result of the test depicted in particular was performed with a colorimetric reader and illumination LEDs with a peak wavelength of 570 nm. A black cassette was used to minimise reflections of all wavelengths, but alternative cassette colours could be used as long as the reflectance of the illumination LEDs is minimised and the absorbance maximised.

The same principle can be applied for a fluorescent reader where the cassette material chosen is known to be minimally fluorescent under the illumination LEDs.

The use of minimally reflective or emissive material in the cassette results in less light reflected off or emitted by the cassette and into the measurement sensors. This helps reduce the effect of light from the ambient environment and prevents the light from the illumination LEDs from straying into neighbouring measurement areas and back into the sensors. Rather it helps prevent light straying from the LED to an adjacent measurement area of the test strip and back to the measurement sensor. Adjacent channel sensors would not normally be active and so should not detect stray light. Furthermore, it contributes to maximising detection of reflection from the test line and reducing background signal noise.

The inventor recognises that the relative intensity of an LED source may be dependent on its forward current. In preferred embodiments, a voltage source arrangement is used to power the illumination LEDs. Because of this voltage source arrangement, the forward current of the LED is affected by both the temperature of the semiconductor die, the diode forward voltage, and the supply voltage, which is typically supplied by a battery. While a more complex current source arrangement would not exhibit these issues, a voltage source arrangement is preferred to minimise complexity and maintain a low-cost design.

The LED die temperature and forward voltage will be dependent on the ambient temperature, frequency of use and current level, such as from the battery supply. Typically, the compensation is calculated and applied by measuring the forward current prior to the start of the test, and then again after. The difference in the forward currents as a ratio may be used by way of appropriate calculations or algorithms in software routines to compensate for any die temperature and battery voltage effects which influence the forward current between the start of the test and when the sample has developed. Applying compensation ensures that the assay measurement results are consistent across the life of the electronic reader. An example of this process is as follows;
(i) Cassette is inserted by the user.
(ii) Forward current of the illumination LEDs are measured and recorded.
(iii) The blank test strip is measured and recorded.
(iv) The user is signalled to apply the sample.
(v) The user applies sample.
(vi) The sample is detected, and the reader waits a predetermined amount of time sufficient to allow for development to occur.
(vii) After the test is complete, the forward current of the illumination LEDs are measured and recorded.
(viii) The developed test strip is measured and the result recorded.
(ix) Using the recorded current and result measurements, a compensated result is calculated.
(x) A compensated result is displayed to the user.

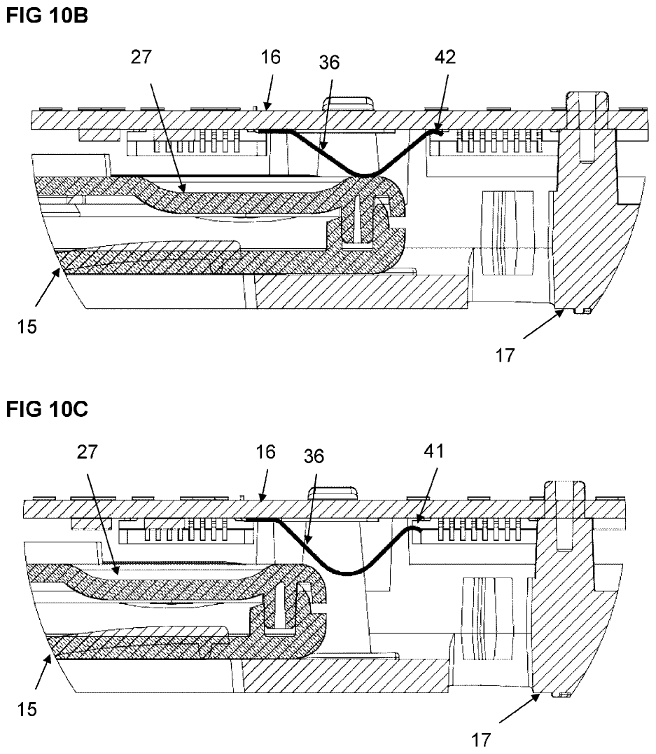

FIG. 10 is a sectional view illustrating operation of a reset switch as a cassette is being inserted into, and removed from, a reader of an embodiment of the present invention showing arrangements in which the reset switch is open (FIG. 10A), closed (FIG. 10C) and re-opened as the cassette is removed from the reader (FIG. 10B). FIG. 10 illustrates the operation of a reset switch 36 as a cassette 15 is inserted into or removed from a reader of the present invention. FIG. 10A and FIG. 10C show the conditions when the reset switch is open while FIG. 10B shows the reset switch 36 is in a closed position. When the cassette 15 is inserted into or removed from the opening in the reader, a normally-open switch is activated which allows the reader to wake-up. This allows the reader to reside in a low power mode while not being used, decreasing the power consumption requirement. This increases the shelf life of the reader. It also has the advantage, compared with the simpler alternative of powering/de-powering the reader using a cassette activated switch, that the reader remains powered after cassette removal—enabling the reader to continue to perform functions such as extended display, communications etc. after cassette removal. It also permits a lower capacity and corresponding less expensive battery to be used.

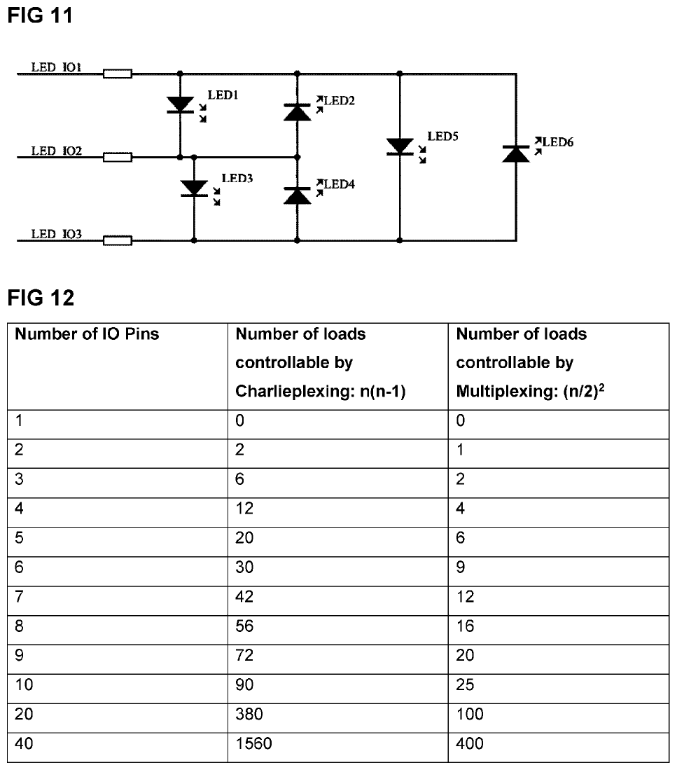

FIG. 11 is a schematic circuit diagram illustrating a basic electronic arrangement according to preferred embodiments of the present invention for the LEDs used in the reader wherein 3 pins controlling 6 LEDs. The remaining 6 LEDs are arranged in a slightly different arrangement, using IO pins 1-3 as well as adding two new pins IO4 and IO5. FIG. 11 illustrates the electronic architecture of a preferred embodiment of the present invention, which allows the use of a greater number of measurement positions and user feedback LEDs than are typically possible with low cost microcontrollers of the prior art.

Typically, in prior art systems, each IO pin controls a single LED. The present invention instead uses a combination of charlieplexing and multiplexing to control multiple LEDs (e.g. twelve, 6 user feedback LEDs and 6 illumination LEDs) from five digital IO pins. Charlieplexing is a multiplexing technique which relies on a combination of the behaviour of LEDs and the tri-state nature of modern microcontroller pins. The IO pins can be High voltage (sourcing current) or Low voltage (sinking current), or High Impedance. A combination of pins being turned between high voltage, low voltage and high impedance can be used to selectively turn on the required LEDs. The critical aspect is that switching occurs on both the high voltage and the low voltage side of a load (normally a load is only switched on either high or low side and not both) and that either side of a load may be positive or negative polarity.

FIG. 12 is a table that shows how charlieplexing and multiplexing can control a very large number of loads as the number of available pins increases. Charlieplexing allows polarity sensitive loads (such as LEDs) to be controlled such that the number of controlled loads is equal to n*(n−1), where n is the number of I/O pins. In comparison, a typical multiplexing arrangement allows for (n/2)² controlled loads to be controlled by n I/O pins.

In a preferred embodiment of the present invention, charlieplexing is used to control the six user feedback LEDs while the remaining 6 LEDs are in a multiplexing arrangement, utilising IO pins 1-3 as well as adding two new pins IO4 and IO5. This is done to accommodate the current measurement and compensation feature as described herein.

This configuration has a disadvantage in that only a single LED can be powered on at once. The restriction is consistent with the desire to have predictable and low current draw from the battery. For this reason, it is preferable to avoid having multiple LEDs on simultaneously.

Furthermore, the design and architecture of this device is such that only a single LED is ever needed to be turned on at any one time. The illumination LEDs are turned on one at a time and the user feedback LEDs are only on when illumination measurements are not occurring. The operation of Measurement and User Feedback LEDs may be interlaced in such a way that multiple User Feedback LEDs may appear to a user to be on simultaneously or such that User Feedback LEDs may appear to be on during measurements but only one LED is ever on. For example, switching two LEDs rapidly on/off so that they both appear on but only one is on at any one time is preferable to having both LEDs on. This way, multiple LEDs may appear to be on when in fact only a single LED is ever switched on at one time.

Multiple LEDs allows the inclusion of other functionality such as a cassette presence/absence detection feature. This feature can be implemented using the LEDs and sensors already provided for user feedback and test illumination. This way the user experience is improved by reducing the number of required interactions prior to performing a test is implemented in software without any additional components.

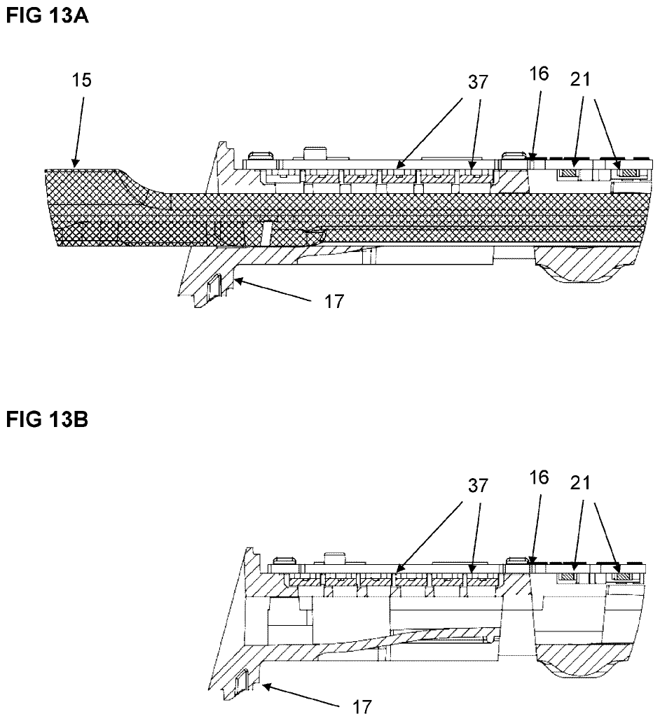

FIG. 13 illustrates one preferred embodiment of the cassette presence/absence detection feature where the user feedback LED 21 closest to the point at which the cassette 15 is inserted in the opening is turned on and measured by the measurement sensor 37 that is also intended for measuring the test strip. The reader can detect when a cassette is inserted in the reader (FIG. 13A) because the light from the user feedback LED is blocked and does not reach the measurement sensor. The reader can also detect the condition when there is no cassette inserted (FIG. 13B), because the light from the user feedback LED reaches the sensor. A threshold in software can be used to determine the presence/absence of a cassette where a low measured signal means a cassette is present and a high measured signal means a cassette is not present. A combination of the reader reset switch and the cassette detection features can be used in software determine what the user intends to do. For example, if the reset switch is toggled and a cassette is detected, it is likely that the user has inserted a cassette and intends to start a test. The alternative scenario is if the reset switch is toggled and there is no cassette detected, then it is likely that the user has just removed a cassette, the powered-on reader can now continue to perform functions such as displaying the result of the previously completed test or maintaining communication with an external device.

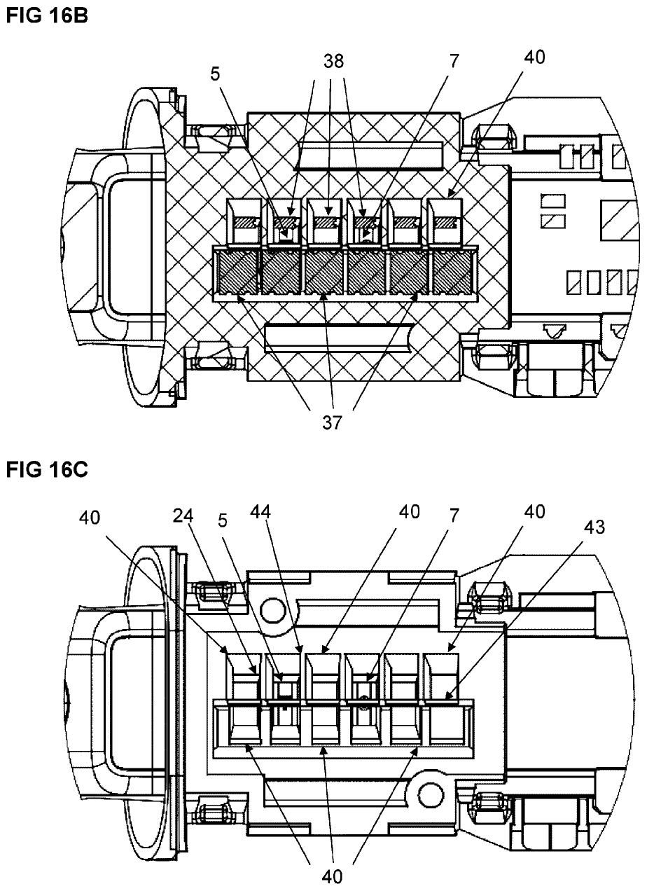

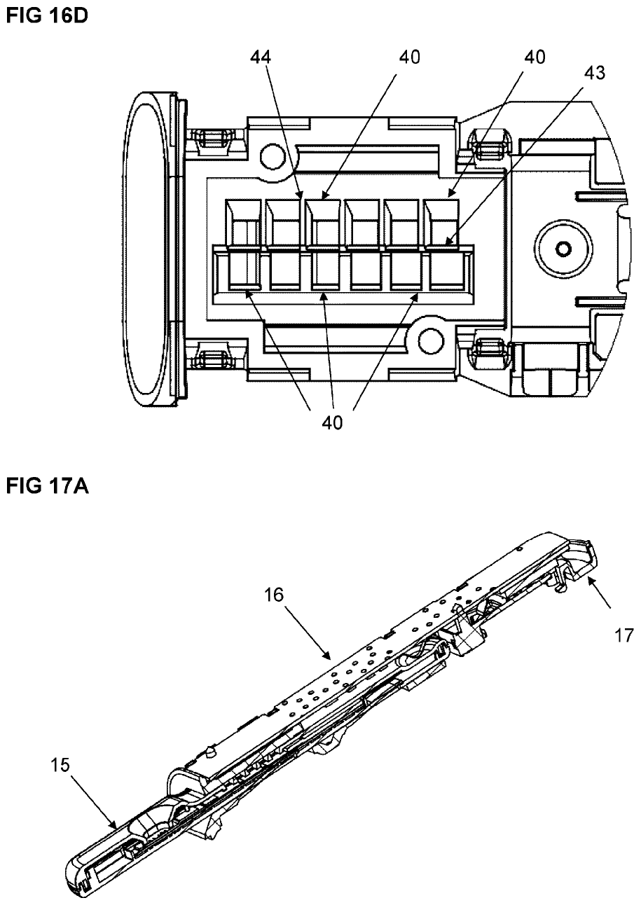

FIG. 14A provides a cross sectional view of a cassette assembly 15 (cassette top 12, cassette bottom 14, and strip 13) inside the reader carrier 17, cross sectioned through an aligned cassette window 24 and carrier window 40, illustrating the separated light guide functionality. The light guide section of the carrier has been synonymously referred to above and herein as the “carrier windows” and the light guide section of the cassette has been synonymously referred to above and herein as the “cassette windows”. The light guide is a functional mask in that restricts the illumination and/or measurable area of the positioned test strip and reduces the refraction and reflection of light to increase the signal to noise ratio. Preferably, the light guide components act mainly as an absorber rather than a refractor or reflector of light. Therefore, in preferred embodiments, light which is reflected from the mask itself is also masked by the differing 3-dimensional structure and positions of the cassette windows 24 and carrier windows 40. The paths of light to or from the illumination LED 38 and to the measurement sensor 37 are shown to be blocked by the separator 43 of carrier windows 40 and cassette top 12 and cassette windows 24. It should be noted that FIG. 14A is essentially a simplified diagram because in reality the light would be bouncing off multiple surfaces. It is also worth noting that the separator 43 as shown is actually part of the carrier window 40 (see FIG. 16 D). FIG. 14B is a detailed view showing how the paths of light to and from the illumination LED 38 and to the measurement sensor 37 fall on the test strip 13, leading to three distinct regions; where the light is incident on the strip 13 but not measured, measurement shadow 45, where the light reaches the strip 13 and is measurable by the sensor 46, and where the sensor may be able to measure but no light reaches, illumination shadow 47. Again, the representation of FIG. 14B is a simplified diagram that implies that there is no light outside of the light paths and 100% of the light is contained within the light path where as in reality the light paths and illumination profile is more complex. FIG. 14C is a simplified top view of the test strip 13 which illustrates how the light guide features ensure that the area framed for measurement 46 in the strip 13 is illuminated and measurable through the cassette window 24, excluding the regions of non-uniform non-specific binding 33.

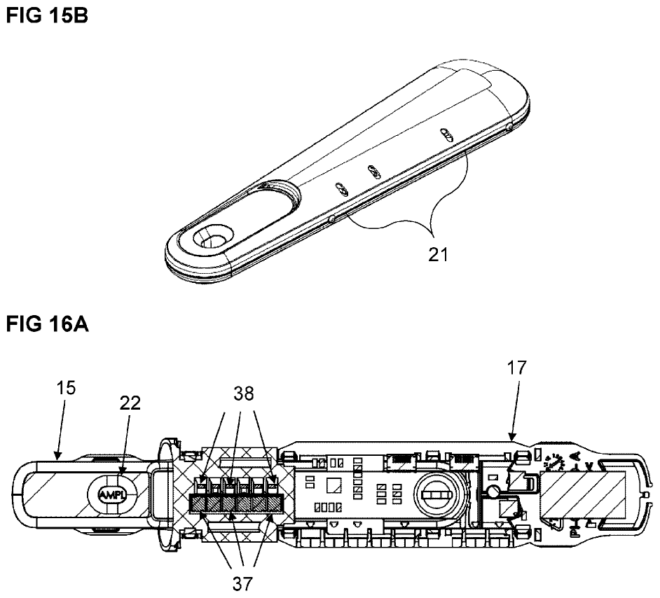

The illustration of FIG. 15A and FIG. 15B depicts a single use version of the lateral flow assay electronic reader of a preferred embodiment of the present invention comprising a PCB 16 with a battery 19, on top of a light guide 50 above a strip 13, encased in a two part housing (top 48 and bottom 49). The PCB 16 holds user feedback LEDs that are visible through holes or apertures 21 in the housing, as shown best in FIG. 15B. A separate light guide 50 is included, which is part of the carrier in the multiuse reader of the other embodiments described herein.

FIG. 16A and FIG. 16B are section views showing an overlay of the LED 38 and sensor 37 locations on top of the carrier 17 and cassette assembly 15. In FIG. 16B the test line 5 and control line 7 are visible. FIG. 16C is a detailed view of the cassette inside the carrier with a clearer view of the individual carrier windows 40 separated by the illumination and sensor separator 43 and adjacent sensor separators 41. The test line 5 and control line 7 are framed by the cassette windows 26 which in turn are framed by the carrier windows 40. FIG. 16D is a detailed view of the carrier windows 40 without the cassette 15 inserted in the carrier 17.

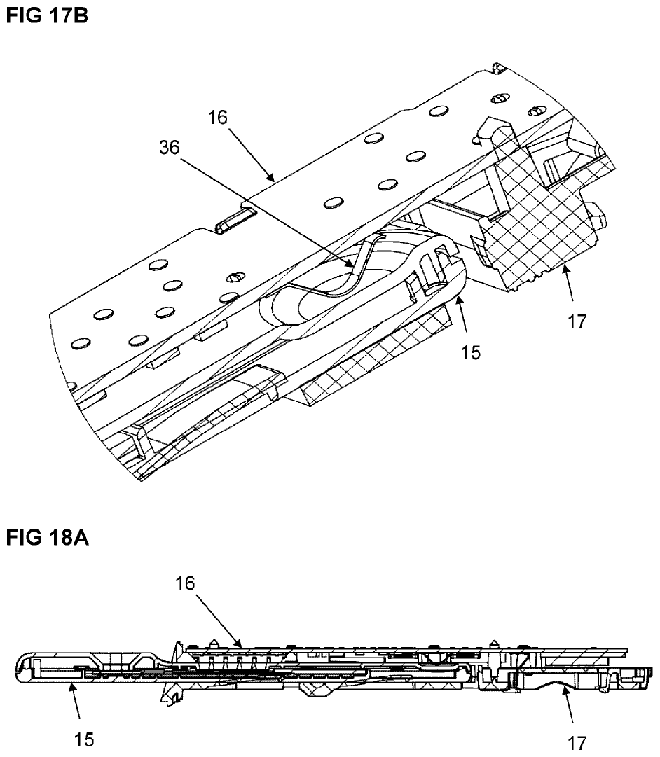

FIG. 17A and FIG. 17B are 3D section views illustrating a cassette 15 fully inserted into a carrier 17 and the reset switch on the PCB 16. It is an alternate view of FIG. 10A.

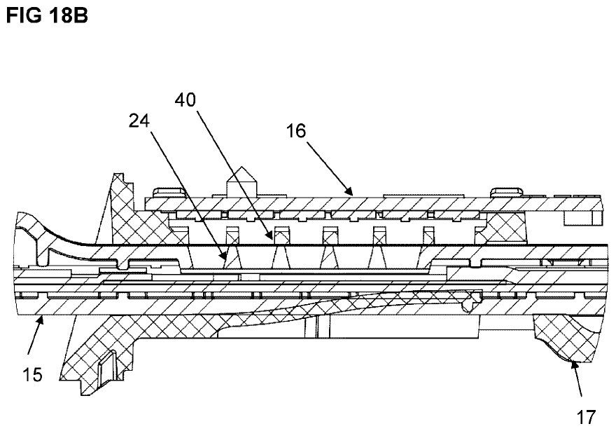

FIG. 18A and FIG. 18B are side section views of a cassette 15 inserted in a carrier 17 showing the alignment of the cassette windows 24 and the carrier windows 40.

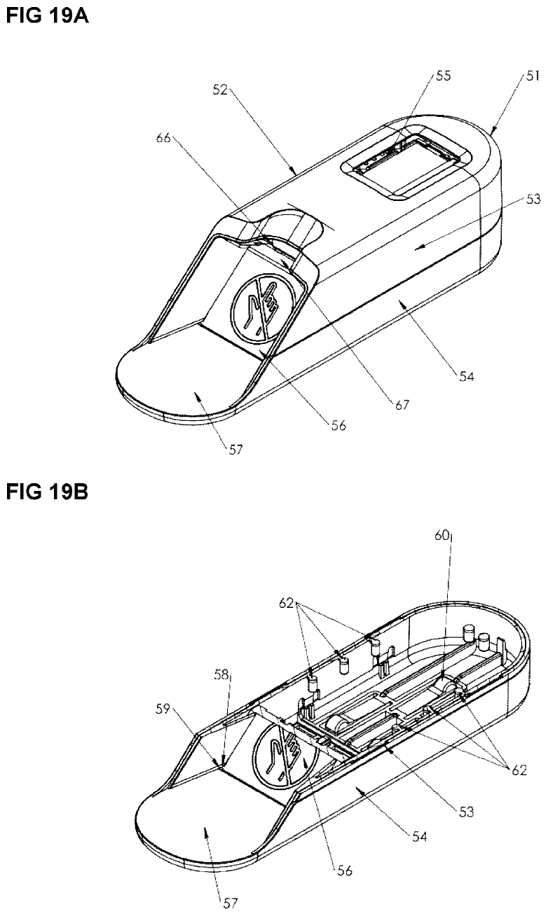

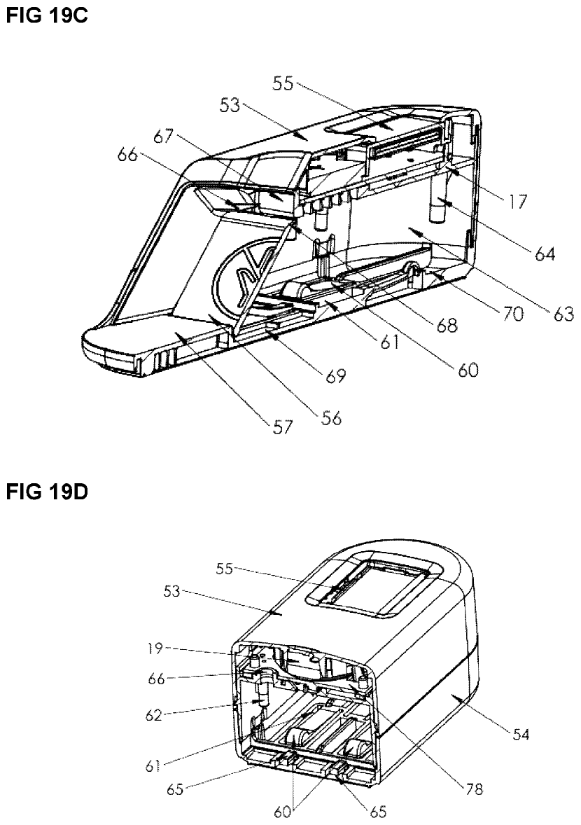

FIGS. 19A, 19B, 19C, 19D, 20A, 20B and 21 show an electronic multiuse reader. The multiuse reader 51 has a reader top 53 and a reader bottom 54 defining a cavity 63 for receiving a cassette 71 with an associated test strip 13. The cavity 63 is further defined by a reader door 56. The reader door 56 may contain an angled lip 68 which interfaces with a lip interface 67 of the carrier 17.

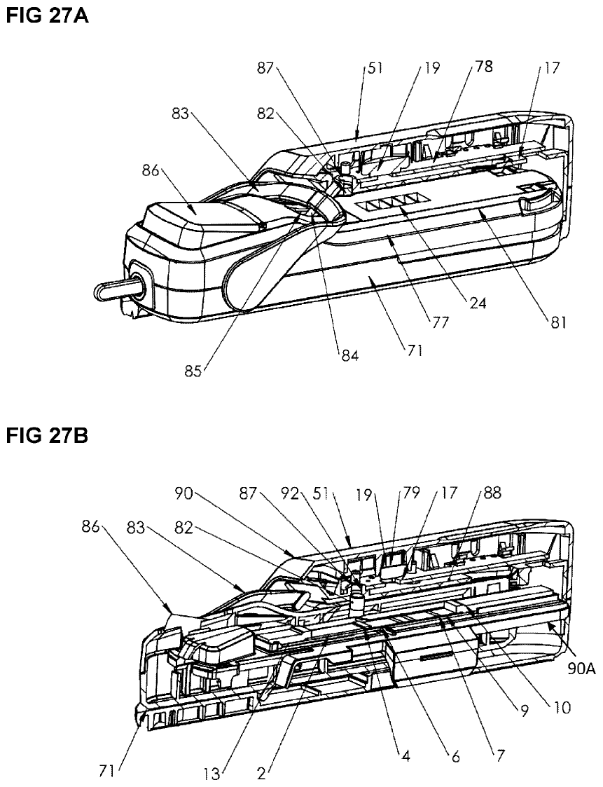

The reader top 53 includes a user interface 55 powered by a battery 19 and controlled by a PCBA 78 mounted to a carrier 17. The carrier 17 comprises a top and side walls. Optionally the carrier further comprises a bottom. The carrier 17 contains carrier windows which are configured to acts as a light guide 88 (see FIGS. 27A-27B) alone or in combination with the cassette windows 24 when a cassette 71 is inserted into the reader 51. The user interface 55 provides a reading of a detected reagent on the test strip 13. At least one end post 64 extends from the carrier 17 into the cavity 63. Locating bosses 62 extend from an under face of the carrier 17 within the reader top 53. The locating bosses 62 preferably extend the full height of the cassette 71.

In an alternate embodiment, the locating bosses 62 can extend into the cavity 63 from the reader bottom 54 or a bottom internal face of the carrier 17. The reader top 53 is preferably rounded.

The reader bottom 54 has an outside reader dock 57 extending to a door receiving section 69 for receiving the reader door 56, and an alignment section 70 with at least one alignment recess 61 and a spring clip 60 or leaf spring. The spring clips 60 are preferably rounded to reduce friction between the bottom of the cassette 71 and the spring clips 60. The door receiving section 69 and the alignment section 70 are within the cavity 63. The reader docket 57 is preferably of a length to support the cassette 71 when it is inserted into the reader 51. The reader bottom 54 is flat for level seating on a surface.

One of the advantages of using a flat reader bottom 54 and a rounded reader top 53 is to encourage placement of the reader and an associated cassette 71 on a flat, level surface, allowing the assay on the test strip 13 of the cassette 71 to run horizontally and prevent temperature changes during measurement by the reader 51.

The reader door 56 has a hinge mechanism in which the door is rotatably attached to the reader 51 by a door pin 58 on either side of the reader door 56 which is received by a reader door socket 59. In an alternate embodiment, a torsion spring can be added to the hinge mechanism.

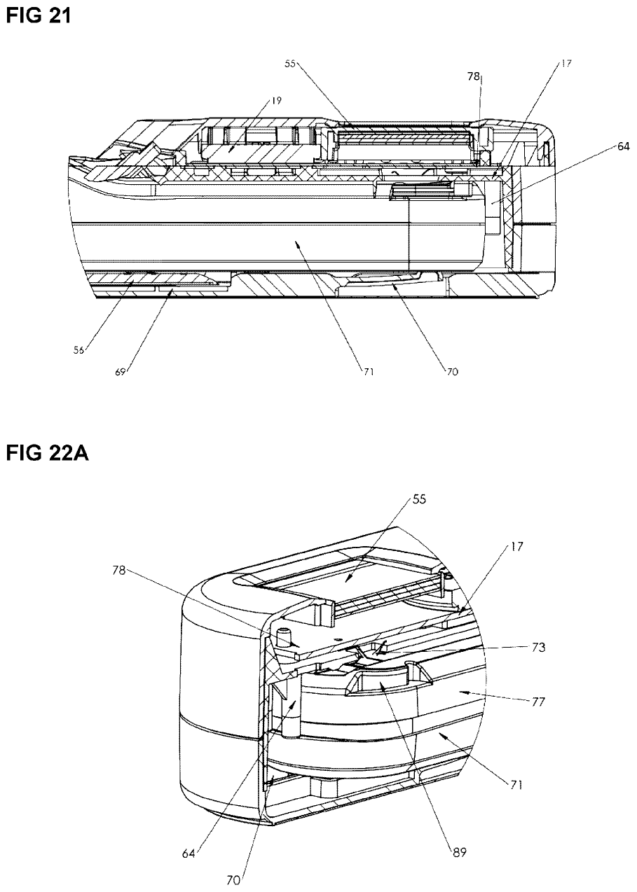

The reader door 56 has a closed position and an open position. In the open position, the reader door 56 rotates such that the reader door 56 is received by the door receiving section 69 of the reader bottom 54, and an external face of the reader door 56 is adjacent to an inserted cassette 71 for example as shown in FIG. 21. In the open position, the door acts to align the cassette 71 within the reader 51, for example by applying a vertical biasing force to the cassette, similar to the vertical biasing springs.

When the reader door is in the closed position, the reader door 56 safeguards internal electronics such as the battery 19 and the PCBA 78, including illumination sources 38 and measurement sensors 37 from dust and other contaminants as well deterring cleaning within the cavity 63 of the reader 51. The reader door 56 is preferably biased towards the closed position by one or more springs 65 located within the reader bottom 54, allowing the door to self-close when the cassette 71 is not present within the reader 51. The one or more springs 65 can interface with one or more recesses (not shown) on the internal back face of the reader door 56. The springs 65 can be made of various materials, such as plastics, metal or other materials which provide resilience and spring force to maintain the reader door 56 in the closed position and allow insertion of a cassette 71 to push the reader door 56 to an open position. The springs can be leaf springs, torsion springs or other springs.

The angle of the reader door 56 within the reader 51 is such that the reader door 56 allow insertion of a cassette 71 to push the reader door 56 to an open position without causing misalignment of the cassette 71 within the cavity of the reader 51. In addition, as the cassette 71 pushes the reader door 56 into the open position, the reader door 56 can be stored within the reader bottom 54 and the cassette 71 slides over the reader door 56 and passes between the lip interface 67 and the reader bottom 54. The lip interface 67 may be part of or integral to the reader top 53 or the carrier 17. The angle of the reader door 56 is such that in the closed position, any gap between the lip interface 67 and the reader door 56 is minimized. The angle of the reader door 56 within the reader 51 is also such that a seal is not necessary. The angle of the reader door 56 is complementary to the lip interface 67 of the carrier 17 to allow mating of the lip 68 of the reader door 56 with the lip interface 67 to prevent liquid, dust, or light to ingress into the reader 51.

While not shown in this embodiment, side rails may be added to the cassette 71 and the reader 51 to increase alignment of two.

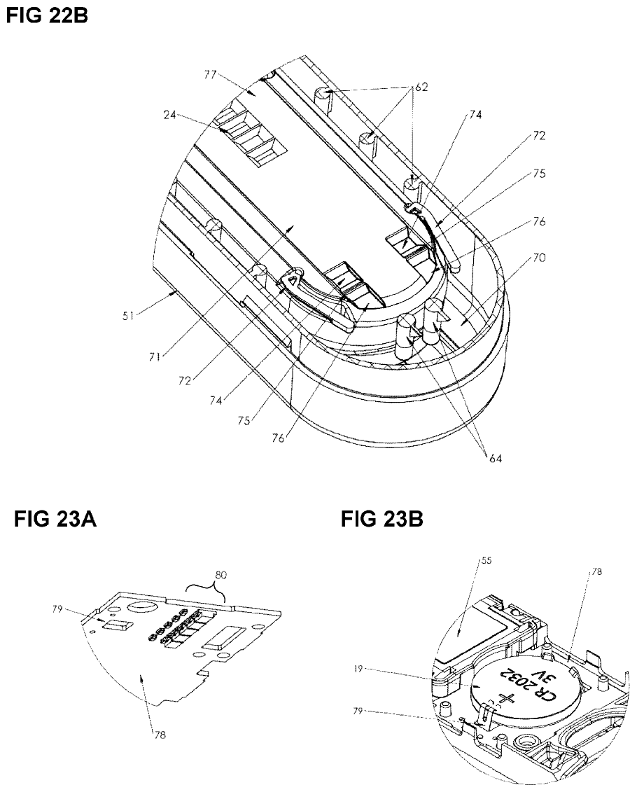

FIG. 22A-22B show sectional views of the cassette 71 inserted into the multiuse reader 51. Soldered to the PCBA 78 is a cassette detection switch 73 which protrudes into the cavity 63 in which the cassette 71 is inserted.

To enable the reader 51 to determine whether or not a cassette 71 is present, a top face of a cassette top 77 has two parallel channels 76 each with a bump 75 and a raised surface 74. In an alternate embodiment, a single channel 76 with a bump 75 and raised surface 74 may be used. As the cassette 71 enters the multiuse reader 51, the bump 75 and the raised surface 74 alternately activate, release and activate the cassette detection switch 73 on insertion and release. Upon removal of the cassette 71, the cassette detection switch 73 is activated and released. The activation of the cassette detection switch 73 wakes up the multiuse reader 51 (from a low power state) and also enables the detection of a cassette 71 in the reader 51, which then triggers the workflow. Since the multiuse reader 51 can be activated upon entry of the cassette 71, the reader 51 can be maintained in a low-power state to conserve battery life when not in use.

An AC coupling circuit interfaces this switch to the microcontroller (MCU) to prevent the MCU from being stuck in its reset (high power) state in the case of partial cassette insertion.

FIG. 23A-23B show close ups of the PCBA 78. The electronics of the PCBA 78 have been designed with low-cost assembly in mind. The PCBA 78 is a two-layer circuit-board with single cycle reflow soldering only. Given that the battery 19 connection is on the opposite side of the board, a custom positive battery terminal 79 is designed to be inserted through the board 78 and soldered on the same side of the board 78 as the rest of the components. By soldering on a single side only, the risk of heat damage due to multiple soldering cycles to sensitive optics components is avoided.

On the bottom side of the PCBA 78 are optics components 80, such as LED 38 and measurement sensors 37, which are used to read the test strip 13. The battery 19 and a liquid crystal display (LCD) (user interface) 55 are located on the top side of the PCBA 78.

It is preferred that the method used to interface the battery 19 to the PCBA does not result in an additional solder cycle. Furthermore, the battery terminal preferably fits through the PCBA 78. The compression force and surface area of the terminal 79 on the battery 19 must ensure reliable connection.

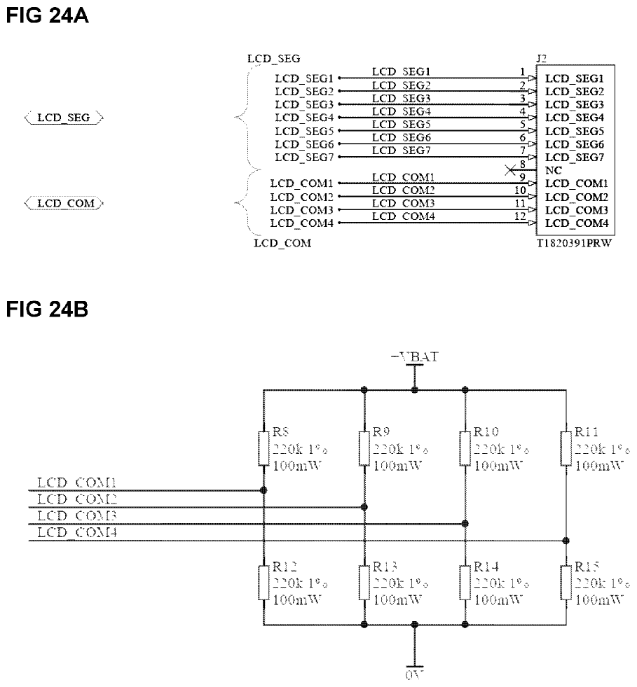

FIG. 24A and 24B refer to circuit diagrams illustrating a simplified architecture to drive a multiplexed LCD arrangement. The arrangement allows for multiplexed LCD drive implementation without a dedicated hardware driver. This arrangement allows simplified architecture to drive a multiplexed LCD directly from a microcontroller without a hardware driver peripheral, using a software driver and external resistor network (R8 to R15).

To display quantitative results, an LCD (see 55) is incorporated into the reader 51. This LCD has a multiplexing ratio of 4. Instead of adding a dedicated hardware driver, the multiplexed LCD segments are driven directly by the microcontroller (MCU) using a software driver. The MCU is already used for other functions in the reader, so no additional integrated circuit is required. By using this arrangement, the number of integrated circuits in the system is reduced, as well the surface area of board space required, allowing a smaller board design and low cost architecture to be used.

To turn an LCD segment On, an AC voltage with a specific root-mean-square threshold voltage must be applied to the segment's electrode. This voltage level for each segment is generated by the MCU in the form of periodic, square waveforms that are either in-phase (segment off) or out-of-phase (segment on). An external resistor ladder is required to set biasing voltage levels.

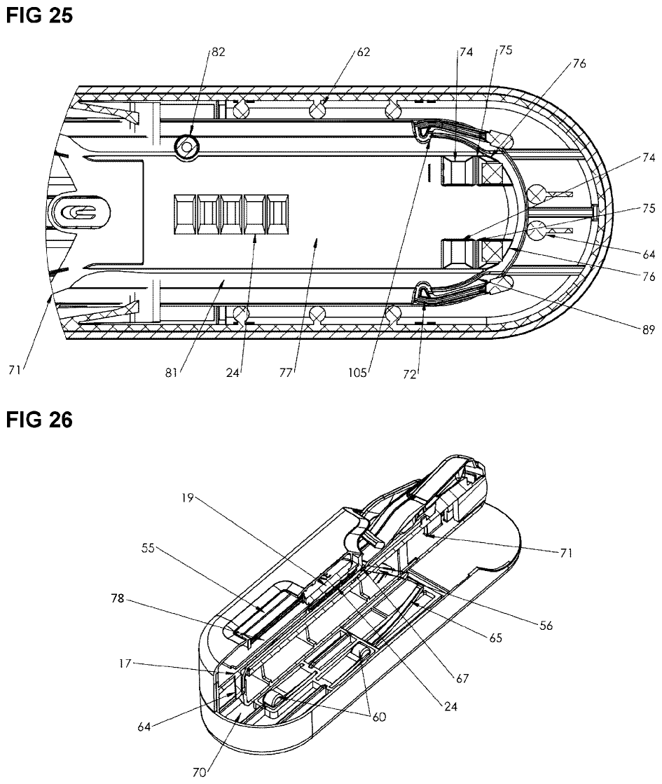

FIGS. 25-27B show alignment and positioning mechanisms for the cassette within the multiuse reader.

To make sure the cassette 71 does not move when the multiuse reader 51 is handled by a user, two retention clips 72 which are attached to or formed as part of the carrier 17, releasably engages with ramp 106 at an end portion of the cassette 71, preferably the cassette top. The retention clips 72 engage with the cassette 71 adjacent the channels 76. At the end portion of the cassette 71 are ramps 89 built into the cassette face 77 for gradual interference with the retention clips 72 followed by sudden engagement of the retention clips 72 when the cassette 71 is fully inserted in the reader 51. For release, the retention clips 72 each have a rounding the face 105 so gradual extraction from the cassette 71 is possible.

The two retention clips 72 each preferably engage with a thin rib 81 of the cassette top 77 surface. The retention clips 72 may also provide haptic feedback to the user when the cassette 71 is fully inserted into the reader 51 as retention clips 72 snap into place onto the cassette 71.

By having the retention clips 72 be a part of or attached to the light guide 88, the retention clips 72 and the positioning and alignment features as described further below present on the same part reduces the tolerance stack. This reduction in tolerance stack, reduces the allowances for tolerances required during manufacture.

The retention clips 72 can also be used pull the cassette 71 into the multiuse reader 51 and maintain an alignment pin 82 of the cassette up against the hard stop 92 of the alignment hole 91 in the reader 51.

An alignment pin 82 is integrally formed with a strip platform 90 which receives the test strip 13 within the cassette 71. The alignment pin 82 extends through the cassette top 77 and can be aligned with a locating or alignment hole 91 of the light guide 88 as well as an alignment boss 87 of the PCBA 79 of the multiuse reader 51. The alignment hole 91 of the light guide 88 has a hard stop 92 which engages with the alignment pin 82 once received within the alignment boss 87 and the alignment hole 91. The alignment hole 91 of the light guide 88 additionally assists with alignment of the electronic components of the reader to the light guide 88 and the test strip 13.

The alignment pin 82 is offset onto one side of the cassette 71 housing so that the alignment pin 82 can extend from the strip platform 90, through the top of the cassette housing without interference with the test strip 13.

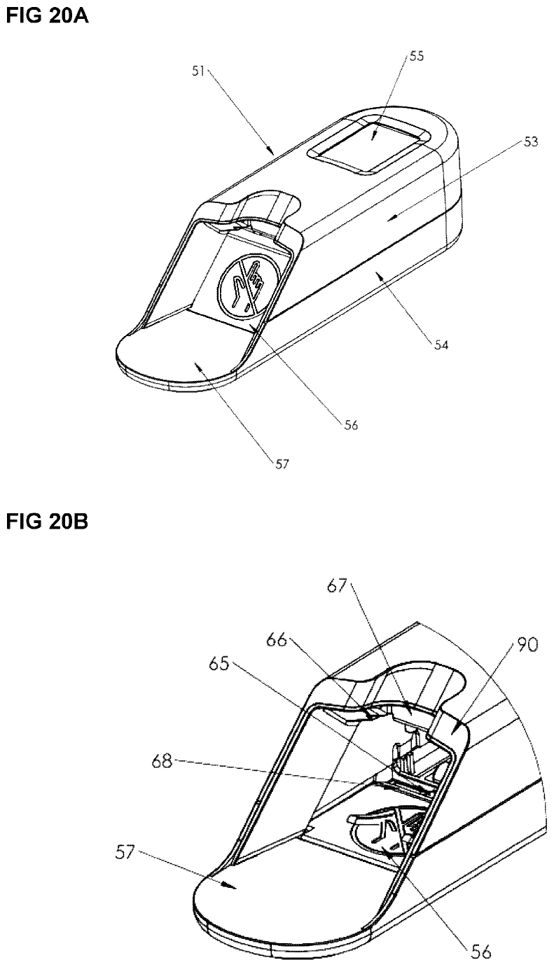

As the cassette 71 is inserted in the reader 51, the interaction of the alignment pin 82 and the hard stop 92 of the alignment hole 91 stops the cassette 71 at the correct position for the alignment of the windows 24 of the cassette 71 the carrier 17, the PCBA and electronics/optics (not shown), and the test strip 13. The U-shaped recess 66 of the lip interface 67 (see FIG. 20B) allows the alignment pin 82 to slide into the reader 51 until the hard stop 92 of the alignment hole 91.

In an alternate embodiment, more than one alignment pin 82 can be used to stop horizontal rotation (to left and right along the horizontal plane) and to reduce tolerance of positioning of the components. In one example, two location pins are provided with one to either side of the windows 24 on the cassette 71 similar to the location pins and posts present in the single use reader of FIG. 15A and 16C.

In another alternate embodiment, the alignment pin 82 may extend from the reader and mate with a recess in the cassette. In this embodiment a rail that the alignment pin could slide in would be present on the cassette.

With the alignment pin 82 holding the cassette 71 in the right position within the alignment hole 91 and alignment boss 87, the cassette 71 would still be able to shift up and down. To ensure vertical alignment, the bottom of the reader 51 has in-built spring features such as spring clips 60 to always push the cassette 71 up onto the bottom surface of the light guide 88. In addition, the thin ribs 81 of the cassette top 77 set the height between the top surface of the cassette 71 and the bottom surface of the light guide 88. This allows the top face of the cassette top 77 with the windows 24, which is slightly recessed, so that the split light guide 88 between the cassette 71 and carrier 17 of the reader 51 do not rub against each other. There is no direct contact between the windows 24 on the cassette 71 and the carrier windows 40. The top face of the cassette top 77 forms a contact with the carrier 17 and assists to block light ingress. This shallow recess is shown, for example in FIG. 14A between carrier 17 and a top surface of cassette top 12. The lack of direct contact of the light guide 88 with windows 24 is important for the multiuse reader 51, as direct contact of the windows 24 of the light guide 88 would result in friction and wearing of the light guides 88 over time as the cassette 71 is insert and removed from the reader 51.

The alignment pin 82 and alignment boss 87 work in conjunction with the locating bosses 62 to reduce the movement of the cassette 71 side to side along the horizontal plane.

The various alignment features described above aid in providing consistent alignment and positioning of the removable cassettes within the reader. Having correct alignment and position of cassettes within the reader reduces errors in reading results, improves variability test to test (reduces reader CV) and improves reader sensitivity.

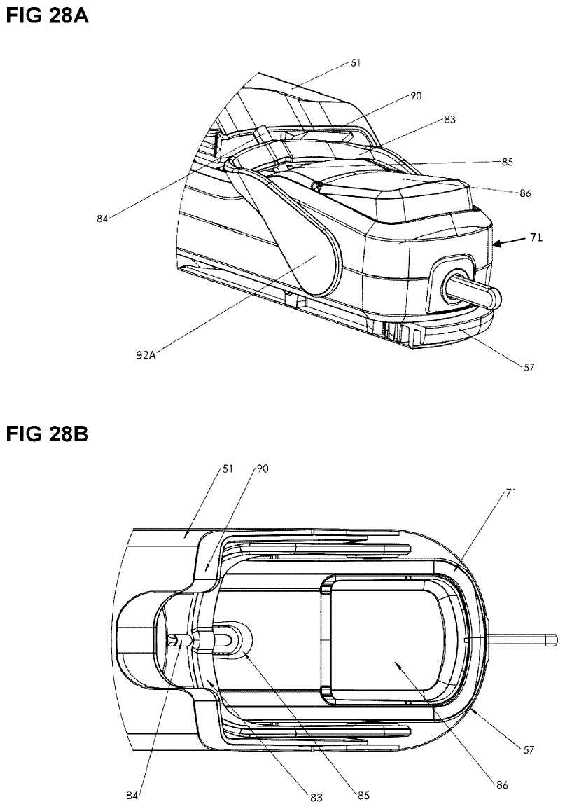

FIG. 28A and 28B show the blood collection unit blocker of the multiuse reader in conjunction with the cassette.

The multiuse reader 51 also preferably has an integral blood collection unit (BCU) blocker 90. The blocker 90 physically prevents rotation of a blood collection unit arm 91 of the cassette 71 from being rotated around an axis 92 after the cassette 71 has been inserted into the multiuse reader 51. The BCU blocker 90 may also assist in blocking light ingress to the reader 51.

In one embodiment, the cassette may be the Pascal RDT Platform from AtomoRapid™ Integrated Rapid Diagnostic Test Platforms of Atomo Diagnostics. Therefore, in order for the multiuse reader 51 to be used, the user has to deposit a sample onto the test strip 13 via the blood collection unit 83 of the cassette 71 prior to insertion of the cassette 71 into the reader 51.

The multiuse reader 51, especially the area close to the blood collection tube 84 of cassette 71 is preferably of a color that visually contrasts highly with blood (e.g. white) and smooth so that a user can do a quick visual check to determine whether there was any blood contamination.

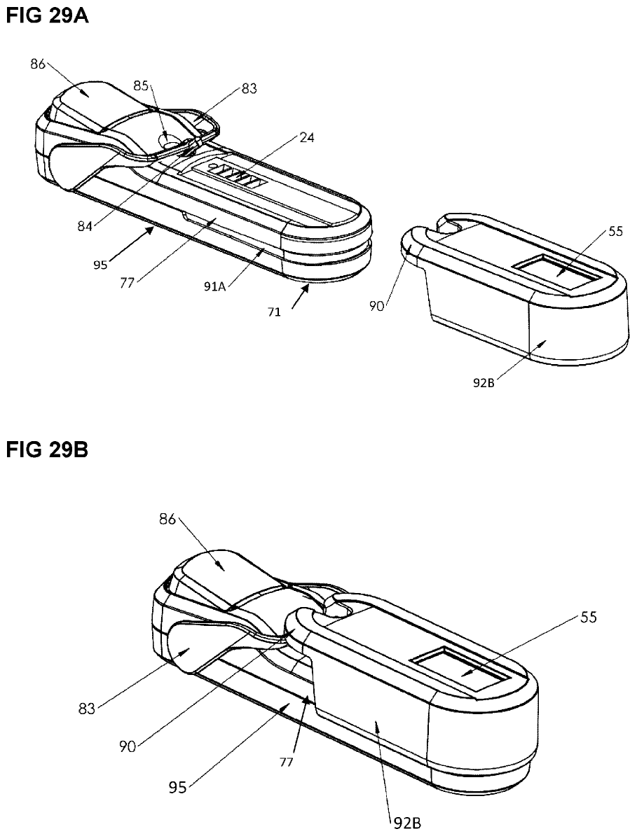

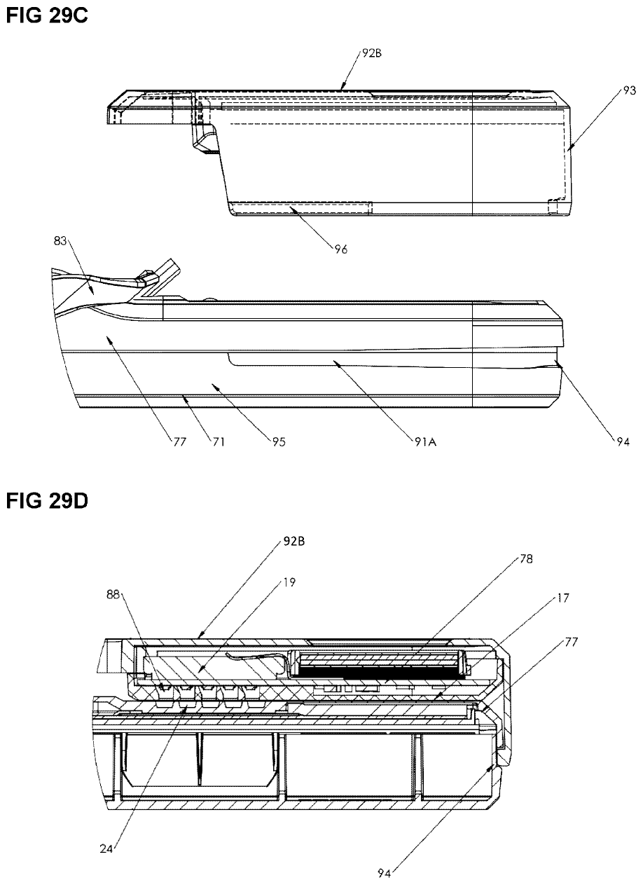

FIG. 29A-29D shows views of a cassette with a slide-on multiuse reader.

Before doing any readings, a sample is collected and deposited, for example by the BCU 83 into the sample port 85 of the cassette 71 and onto the test strip 13 by rotating the BCU 83. This cannot be done after the reader 92 has been put into place.

The slide-on multiuse reader 92 can be slid onto the cassette 71 to read test strip results by aligning a sliding feature of the reader 92 with mating or corresponding rails 91 or another sliding feature in a cassette bottom 95. The rail 91 may be located on a split line between the cassette top 77 and the cassette bottom 95 or another place on the cassette 71. The rails and sliding feature additionally facilitate high precision alignment between the cassette 71 and the reader 92.

When the cassette is in place within the reader 92, a shroud 93 of the reader 92 is formed to block out light.

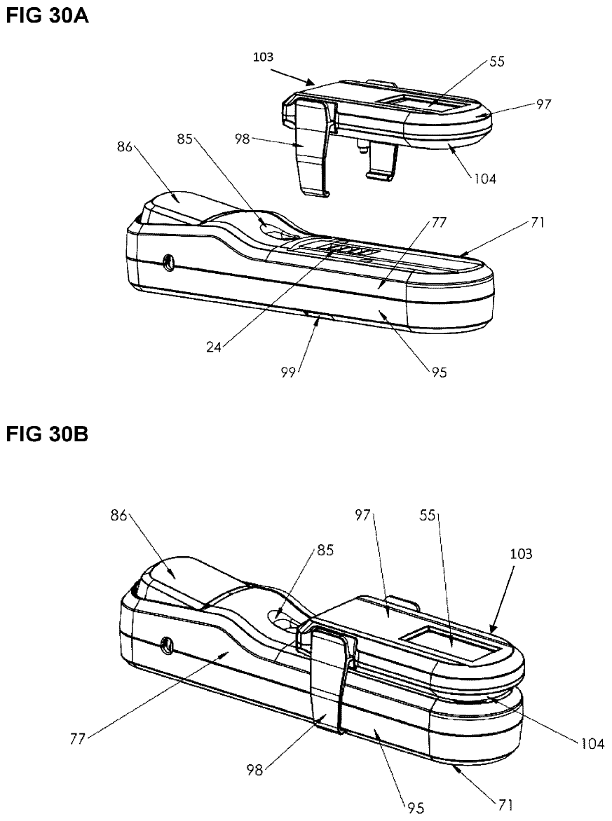

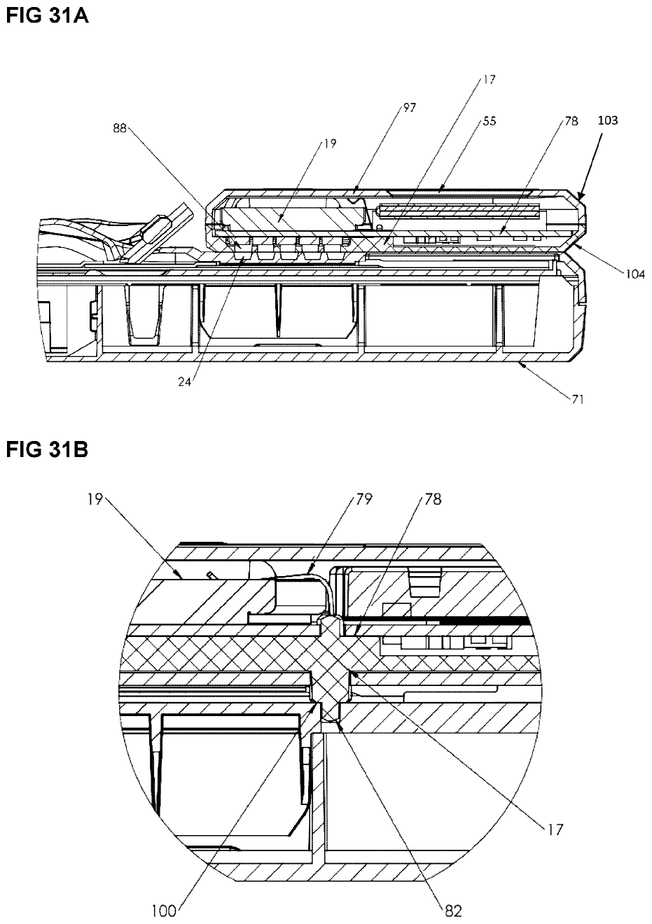

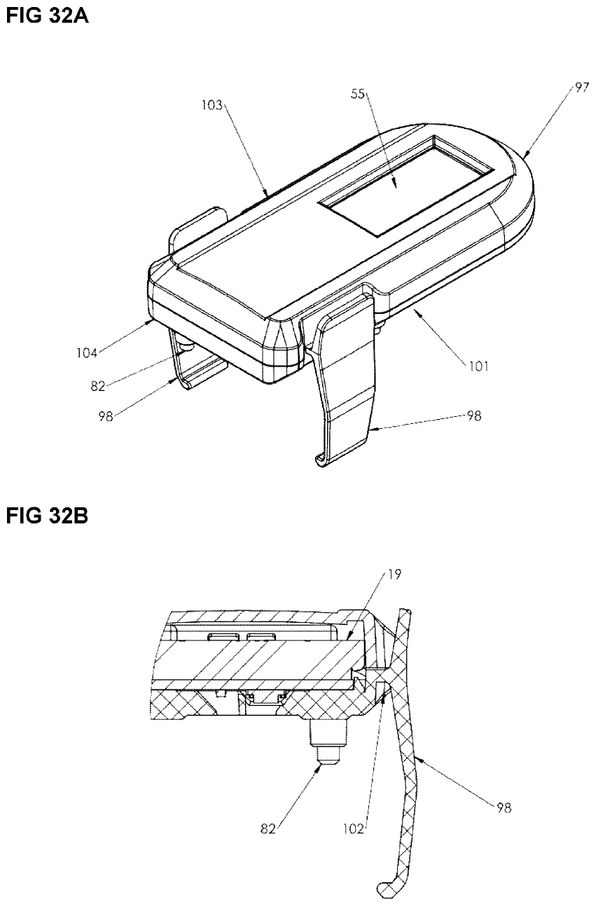

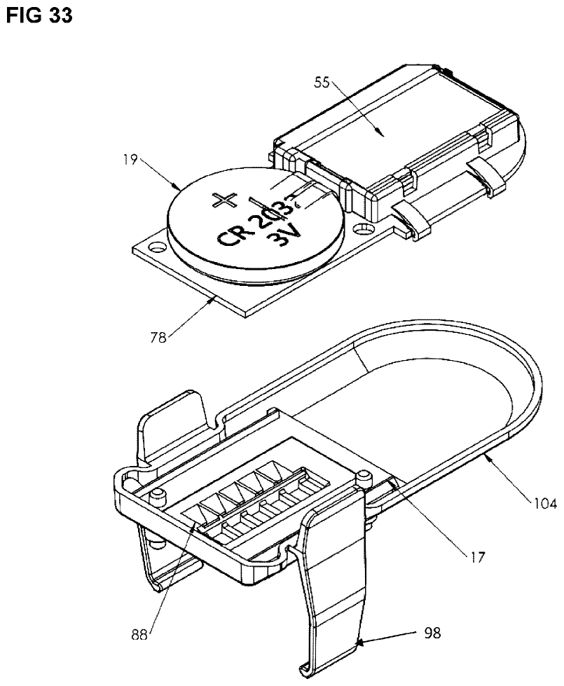

FIG. 30A-33 show a clip-on multiuse reader. The clip-on multiuse reader 97 has a reader top 97 attached to a reader bottom 104. The reader top 97 has a user interface 55. The reader bottom 104 has clips 98 which are attached via plastic hinge 102. An alignment pin 82 extends outwards from the reader bottom 104. Within the clip-on multiuse reader 97 includes a PCBA 79 with a battery 19 on a top surface. The reader bottom 104 has a carrier 17 with a light guide 88.

A clip-on multiuse reader 97 can be clipped on to the cassette 71 by the clips 98 of the reader 97 which are received by a recess 99 on the cassette bottom 95. The recess 99 is aligned with the windows 24 such that the light guide 88 of the reader bottom 104 is aligned with the windows 24 when the clip-on multiuse reader 97 is clipped onto the cassette 71. To help with the alignment, at least one alignment pin 82 is received within a recess on the cassette top 77. A shoulder 100 of the alignment pin 82 sets the height between the cassette 71 and the reader 97.

In an alternate embodiment, the cassette windows 24 may be combined with the light guide 88 (carrier windows 40) such that both sets of the windows 24 and 40 that feature as a split light guide are formed as part of the carrier 17. In this configuration, the cassette top 77 comprises a single window. As the clip-on reader is clipped down from the top face of the cassette, rather than sliding onto the cassette, the light guide features of the carrier can protrude out from the reader and can fit the form of the cassette window. The protruding light guide comprises the advantages of the split light guide in a single component taking the features of the light guide to the surface of the test strip and extend into the reader electronics (LEDs and detectors). In this configuration, the cassette top 77 provides a clear view of the test strip 13 when the reader is not attached, thereby allowing the user to visually determine the test result without the use of a reader.

The clip-on multiuse reader 97 can be removed from the cassette by squeezing the clips 98, allowing the clips 98 to pivot on hinge 102.

While this invention has been described in connection with specific embodiments thereof, it will be understood that it is capable of further modification(s). This application is intended to cover any variations uses or adaptations of the invention following in general, the principles of the invention and including such departures from the present disclosure as come within known or customary practice within the art to which the invention pertains and as may be applied to the essential features hereinbefore set forth.

As the present invention may be embodied in several forms without departing from the spirit of the essential characteristics of the invention, it should be understood that the above described embodiments are not to limit the present invention unless otherwise specified, but rather should be construed broadly within the spirit and scope of the invention as defined in the appended claims. The described embodiments are to be considered in all respects as illustrative only and not restrictive.

Various modifications and equivalent arrangements are intended to be included within the spirit and scope of the invention and appended claims. Therefore, the specific embodiments are to be understood to be illustrative of the many ways in which the principles of the present invention may be practiced. In the following claims, any means-plus-function clauses are intended to cover structures as performing the defined function and not only structural equivalents, but also equivalent structures. For example, although a nail and a screw may not be structural equivalents in that a nail employs a cylindrical surface to secure wooden parts together, whereas a screw employs a helical surface to secure wooden parts together, in the environment of fastening wooden parts, a nail and a screw are equivalent structures.
The following sections I-VII provide a guide to interpreting the present specification.

### I. Terms

Different industry sectors and different countries use varying terminology to describe lateral flow assay products and devices. Some commonly used names include but are not limited to Lateral flow test (LFT), Lateral flow device (LFD), Lateral flow assay (LFA), Lateral flow immunoassay (LFIA), Lateral flow immunochromatographic assays, Dipstick, Pen-side test, Quick test, Rapid test, and Test strip. Accordingly, the present invention is not limited by any particular embodiment of lateral flow assay.

The term “sensor” is to be taken as synonymous with the terms “measurement sensor” or “illumination sensor”.

The term “result line”, “result lines” or “test result line” means the regions of the test strip where there are capture antibodies placed. These regions typically develop into test lines or control lines.

The term “test background” refers to a region of a test strip that is proximate or adjacent a result line or test line and which may be included in the regions of the test strip that are detected by the electronic lateral flow assay test reader.

The term “strip background” refers to a region of a test strip without capture antibodies and which is not included in the regions of the test strip that are detected by the electronic lateral flow assay test reader when detecting a result line.

The term “minimally reflective” means an attribute of the material that is configured to an illumination source wavelength in order to minimise the light reflected or emitted from the material.

The term a “viewing area” means one or more windows on the cassette.

The term “measurement area” means one or more windows on the reader.

The term “development area” means the area of the test strip where the test and/or control lines may develop. The development area can also comprise at least one area forming part or all of the strip background region

The term “test strip” is used herein in reference to the strip of material(s) utilised for a lateral flow assay test, which may comprise one or a combination of a sample pad, conjugate pad, a capillary bed having a development area, which itself may include zones comprising test and control zones inclusive of test and control lines, background regions, and a waste pad. Where the context of the description herein requires, the term is used for particular reference to the development area of the test strip.

The term “tolerance stack” would be appreciated by the person skilled in the art and is reference to the accumulation of error or uncertainty in a dimension due to uncertainty in each of a number of separate components or relationships. Accordingly, it may be considered the sum of uncertainties which make up the total uncertainty in a dimension.

The term “product” means any machine, manufacture and/or composition of matter, unless expressly specified otherwise.

The term “process” means any process, algorithm, method or the like, unless expressly specified otherwise.

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

The term “invention” and the like mean “the one or more inventions disclosed in this specification”, unless expressly specified otherwise.

The terms “an embodiment”, “embodiment”, “embodiments”, “the embodiment”, “the embodiments”, “one or more embodiments”, “some embodiments”, “certain embodiments”, “one embodiment”, “another embodiment” and the like mean “one or more (but not all) embodiments of the disclosed invention(s)”, unless expressly specified otherwise.

The term “variation” of an invention means an embodiment of the invention, unless expressly specified otherwise.

A reference to “another embodiment” in describing an embodiment does not imply that the referenced embodiment is mutually exclusive with another embodiment (e.g., an embodiment described before the referenced embodiment), unless expressly specified otherwise.

The terms “including”, “comprising” and variations thereof mean “including but not limited to”, unless expressly specified otherwise.

The terms “a”, “an” and “the” mean “one or more”, unless expressly specified otherwise.

The term “plurality” means “two or more”, unless expressly specified otherwise.

The term “herein” means “in the present specification, including anything which may be incorporated by reference”, unless expressly specified otherwise.

The phrase “at least one of”, when such phrase modifies a plurality of things (such as an enumerated list of things), means any combination of one or more of those things, unless expressly specified otherwise. For example, the phrase “at least one of a widget, a car and a wheel” means either (i) a widget, (ii) a car, (iii) a wheel, (iv) a widget and a car, (v) a widget and a wheel, (vi) a car and a wheel, or (vii) a widget, a car and a wheel. The phrase “at least one of”, when such phrase modifies a plurality of things, does not mean “one of each of” the plurality of things.

Numerical terms such as “one”, “two”, etc. when used as cardinal numbers to indicate quantity of something (e.g., one widget, two widgets), mean the quantity indicated by that numerical term, but do not mean at least the quantity indicated by that numerical term. For example, the phrase “one widget” does not mean “at least one widget”, and therefore the phrase “one widget” does not cover, e.g., two widgets.

The phrase “based on” does not mean “based only on”, unless expressly specified otherwise. In other words, the phrase “based on” describes both “based only on” and “based at least on”. The phrase “based at least on” is equivalent to the phrase “based at least in part on”.

The term “represent” and like terms are not exclusive, unless expressly specified otherwise. For example, the term “represents” do not mean “represents only”, unless expressly specified otherwise. In other words, the phrase “the data represents a credit card number” describes both “the data represents only a credit card number” and “the data represents a credit card number and the data also represents something else”.

The term “whereby” is used herein only to precede a clause or other set of words that express only the intended result, objective or consequence of something that is previously and explicitly recited. Thus, when the term “whereby” is used in a claim, the clause or other words that the term “whereby” modifies do not establish specific further limitations of the claim or otherwise restricts the meaning or scope of the claim.

The term “e.g.” and like terms mean “for example”, and thus does not limit the term or phrase it explains. For example, in the sentence “the computer sends data (e.g., instructions, a data structure) over the Internet”, the term “e.g.” explains that “instructions” are an example of “data” that the computer may send over the Internet, and also explains that “a data structure” is an example of “data” that the computer may send over the Internet. However, both “instructions” and “a data structure” are merely examples of “data”, and other things besides “instructions” and “a data structure” can be “data”.

The term “i.e.” and like terms mean “that is”, and thus limits the term or phrase it explains. For example, in the sentence “the computer sends data (i.e., instructions) over the Internet”, the term “i.e.” explains that “instructions” are the “data” that the computer sends over the Internet.

Any given numerical range shall include whole and fractions of numbers within the range. For example, the range “1 to 10” shall be interpreted to specifically include whole numbers between 1 and 10 (e.g., 2, 3, 4, . . . 9) and non-whole numbers (e.g., 1.1, 1.2, . . . 1.9).

### II. Determining

The term “determining” and grammatical variants thereof (e.g., to determine a price, determining a value, determine an object which meets a certain criterion) is used in an extremely broad sense. The term “determining” encompasses a wide variety of actions and therefore “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing, and the like.

The term “determining” does not imply certainty or absolute precision, and therefore “determining” can include estimating, extrapolating, predicting, guessing and the like.

The term “determining” does not imply that mathematical processing must be performed, and does not imply that numerical methods must be used, and does not imply that an algorithm or process is used.

The term “determining” does not imply that any particular device must be used. For example, a computer need not necessarily perform the determining.

### III. Indication

The term “indication” is used in an extremely broad sense. The term “indication” may, among other things, encompass a sign, symptom, or token of something else.

The term “indication” may be used to refer to any indicia and/or other information indicative of or associated with a subject, item, entity, and/or other object and/or idea.

As used herein, the phrases “information indicative of” and “indicia” may be used to refer to any information that represents, describes, and/or is otherwise associated with a related entity, subject, or object.

Indicia of information may include, for example, a symbol, a code, a reference, a link, a signal, an identifier, and/or any combination thereof and/or any other informative representation associated with the information.

In some embodiments, indicia of information (or indicative of the information) may be or include the information itself and/or any portion or component of the information. In some embodiments, an indication may include a request, a solicitation, a broadcast, and/or any other form of information gathering and/or dissemination.

### IV. Forms of Sentences

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device or article is described herein, more than one device/article (whether or not they cooperate) may alternatively be used in place of the single device/article that is described. Accordingly, the functionality that is described as being possessed by a device may alternatively be possessed by more than one device/article (whether or not they cooperate).

Similarly, where more than one device or article is described herein (whether or not they cooperate), a single device/article may alternatively be used in place of the more than one device or article that is described. For example, a plurality of computer-based devices may be substituted with a single computer-based device. Accordingly, the various functionality that is described as being possessed by more than one device or article may alternatively be possessed by a single device/article.

The functionality and/or the features of a single device that is described may be alternatively embodied by one or more other devices which are described but are not explicitly described as having such functionality/features. Thus, other embodiments need not include the described device itself, but rather can include the one or more other devices which would, in those other embodiments, have such functionality/features.

### V. Disclosed Examples and Terminology Are Not Limiting

Neither the Title nor the Abstract in this specification is intended to be taken as limiting in any way as the scope of the disclosed invention(s). The title and headings of sections provided in the specification are for convenience only, and are not to be taken as limiting the disclosure in any way.

Numerous embodiments are described in the present application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognise that the disclosed invention(s) may be practised with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is not a literal description of all embodiments of the invention(s). Also, the present disclosure is not a listing of features of the invention(s) which must be present in all embodiments.

Devices that are described as in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for long period of time (e.g. weeks at a time). In addition, devices that are in communication with each other may communicate directly or indirectly through one or more intermediaries.

A description of an embodiment with several components or features does not imply that all or even any of such components/features are required. On the contrary, a variety of optional components are described to illustrate the wide variety of possible embodiments of the present invention(s). Unless otherwise specified explicitly, no component/feature is essential or required.

Although process steps, operations, algorithms or the like may be described in a particular sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention(s), and does not imply that the illustrated process is preferred.

Although a process may be described as including a plurality of steps, that does not imply that all or any of the steps are preferred, essential or required. Various other embodiments within the scope of the described invention(s) include other processes that omit some or all of the described steps. Unless otherwise specified explicitly, no step is essential or required.

Although a process may be described singly or without reference to other products or methods, in an embodiment the process may interact with other products or methods. For example, such interaction may include linking one business model to another business model. Such interaction may be provided to enhance the flexibility or desirability of the process.

Although a product may be described as including a plurality of components, aspects, qualities, characteristics and/or features, that does not indicate that any or all of the plurality are preferred, essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

An enumerated list of items (which may or may not be numbered) does not imply that any or all of the items are equivalent to each other or readily substituted for each other.

All embodiments are illustrative, and do not imply that the invention or any embodiments were made or performed, as the case may be.

### VI. Computing

It will be readily apparent to one of ordinary skill in the art that the various processes described herein may be implemented by, e.g., appropriately programmed general purpose computers, special purpose computers and computing devices. Typically a processor (e.g., one or more microprocessors, one or more micro-controllers, one or more digital signal processors) will receive instructions (e.g., from a memory or like device), and execute those instructions, thereby performing one or more processes defined by those instructions.

A “processor” means one or more microprocessors, central processing units (CPUs), computing devices, micro-controllers, digital signal processors, or like devices or any combination thereof.

Thus a description of a process is likewise a description of an apparatus for performing the process. The apparatus that performs the process can include, e.g., a processor and those input devices and output devices that are appropriate to perform the process.

Further, programs that implement such methods (as well as other types of data) may be stored and transmitted using a variety of media (e.g., computer readable media) in a number of manners. In some embodiments, hard-wired circuitry or custom hardware may be used in place of, or in combination with, some or all of the software instructions that can implement the processes of various embodiments. Thus, various combinations of hardware and software may be used instead of software only.

The term “computer-readable medium” refers to any medium, a plurality of the same, or a combination of different media, that participate in providing data (e.g., instructions, data structures) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fibre optics, including the wires that comprise a system bus coupled to the processor. Transmission media may include or convey acoustic waves, light waves and electromagnetic emissions, such as those generated during radio frequency (RF) and infra-red (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

Various forms of computer readable media may be involved in carrying data (e.g. sequences of instructions) to a processor. For example, data may be (i) delivered from RAM to a processor; (ii) carried over a wireless transmission medium; (iii) formatted and/or transmitted according to numerous formats, standards or protocols, such as Ethernet (or IEEE 802.3), SAP, ATP, Bluetooth™, and TCP/IP, TDMA, CDMA, and 3G; and/or (iv) encrypted to ensure privacy or prevent fraud in any of a variety of ways well known in the art.

Thus, a description of a process is likewise a description of a computer-readable medium storing a program for performing the process. The computer-readable medium can store (in any appropriate format) those program elements which are appropriate to perform the method.

Just as the description of various steps in a process does not indicate that all the described steps are required, embodiments of an apparatus include a computer/computing device operable to perform some (but not necessarily all) of the described process.

Likewise, just as the description of various steps in a process does not indicate that all the described steps are required, embodiments of a computer-readable medium storing a program or data structure include a computer-readable medium storing a program that, when executed, can cause a processor to perform some (but not necessarily all) of the described process.

Where databases are described, it will be understood by one of ordinary skill in the art that (i) alternative database structures to those described may be readily employed, and (ii) other memory structures besides databases may be readily employed. Any illustrations or descriptions of any sample databases presented herein are illustrative arrangements for stored representations of information. Any number of other arrangements may be employed besides those suggested by, e.g., tables illustrated in drawings or elsewhere. Similarly, any illustrated entries of the databases represent exemplary information only; one of ordinary skill in the art will understand that the number and content of the entries can be different from those described herein. Further, despite any depiction of the databases as tables, other formats (including relational databases, object-based models and/or distributed databases) could be used to store and manipulate the data types described herein. Likewise, object methods or behaviours of a database can be used to implement various processes, such as the described herein. In addition, the databases may, in a known manner, be stored locally or remotely from a device which accesses data in such a database.

Various embodiments can be configured to work in a network environment including a computer that is in communication (e.g., via a communications network) with one or more devices. The computer may communicate with the devices directly or indirectly, via any wired or wireless medium (e.g. the Internet, LAN, WAN or Ethernet, Token Ring, a telephone line, a cable line, a radio channel, an optical communications line, commercial on-line service providers, bulletin board systems, a satellite communications link, a combination of any of the above). Each of the devices may themselves comprise computers or other computing devices that are adapted to communicate with the computer. Any number and type of devices may be in communication with the computer.

In an embodiment, a server computer or centralised authority may not be necessary or desirable. For example, the present invention may, in an embodiment, be practised on one or more devices without a central authority. In such an embodiment, any functions described herein as performed by the server computer or data described as stored on the server computer may instead be performed by or stored on one or more such devices.

Where a process is described, in an embodiment the process may operate without any user intervention. In another embodiment, the process includes some human intervention (e.g., a step is performed by or with the assistance of a human).

It should be noted that where the terms “server”, “secure server” or similar terms are used herein, a communication device is described that may be used in a communication system, unless the context otherwise requires, and should not be construed to limit the present invention to any particular communication device type. Thus, a communication device may include, without limitation, a bridge, router, bridge-router (router), switch, node, or other communication device, which may or may not be secure.

It should also be noted that where a flowchart is used herein to demonstrate various aspects of the invention, it should not be construed to limit the present invention to any particular logic flow or logic implementation. The described logic may be partitioned into different logic blocks (e.g., programs, modules, functions, or subroutines) without changing the overall results or otherwise departing from the true scope of the invention. Often, logic elements may be added, modified, omitted, performed in a different order, or implemented using different logic constructs (e.g., logic gates, looping primitives, conditional logic, and other logic constructs) without changing the overall results or otherwise departing from the true scope of the invention.

Various embodiments of the invention may be embodied in many different forms, including computer program logic for use with a processor (e.g., a microprocessor, microcontroller, digital signal processor, or general purpose computer and for that matter, any commercial processor may be used to implement the embodiments of the invention either as a single processor, serial or parallel set of processors in the system and, as such, examples of commercial processors include, but are not limited to Merced™, Pentium™, Pentium II™, Xeon™, Celeron™, Pentium Pro™, Efficeon™, Athlon™, AMD™ and the like), programmable logic for use with a programmable logic device (e.g., a Field Programmable Gate Array (FPGA) or other PLD), discrete components, integrated circuitry (e.g., an Application Specific Integrated Circuit (ASIC)), or any other means including any combination thereof. In an exemplary embodiment of the present invention, predominantly all of the communication between users and the server is implemented as a set of computer program instructions that is converted into a computer executable form, stored as such in a computer readable medium, and executed by a microprocessor under the control of an operating system.

Computer program logic implementing all or part of the functionality where described herein may be embodied in various forms, including a source code form, a computer executable form, and various intermediate forms (e.g., forms generated by an assembler, compiler, linker, or locator). Source code may include a series of computer program instructions implemented in any of various programming languages (e.g., an object code, an assembly language, or a high-level language such as Fortran, C, C++, JAVA, or HTML. Moreover, there are hundreds of available computer languages that may be used to implement embodiments of the invention, among the more common being Ada; Algol; APL; awk; Basic; C; C++; Conol; Delphi; Eiffel; Euphoria; Forth; Fortran; HTML; Icon; Java; Javascript; Lisp; Logo; Mathematica; MatLab; Miranda; Modula-2; Oberon; Pascal; Perl; PL/I; Prolog; Python; Rexx; SAS; Scheme; sed; Simula; Smalltalk; Snobol; SQL; Visual Basic; Visual C++; Linux and XML.) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g, a RAM, ROM, PROM, EEPROM, or Flash-Programmable RAM), a magnetic memory device (e.g., a diskette or fixed disk), an optical memory device (e.g., a CD-ROM or DVD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies (e.g., Bluetooth), networking technologies, and inter-networking technologies. The computer program may be distributed in any form as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web).

Hardware logic (including programmable logic for use with a programmable logic device) implementing all or part of the functionality where described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL). Hardware logic may also be incorporated into display screens for implementing embodiments of the invention and which may be segmented display screens, analogue display screens, digital display screens, CRTs, LED screens, Plasma screens, liquid crystal diode screen, and the like.

Programmable logic may be fixed either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM or DVD-ROM), or other memory device. The programmable logic may be fixed in a signal that is transmittable to a computer using any of various communication technologies, including, but in no way limited to, analog technologies, digital technologies, optical technologies, wireless technologies (e.g., Bluetooth), networking technologies, and internetworking technologies. The programmable logic may be distributed as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web).

“Comprises/comprising” and “includes/including” when used in this specification is taken to specify the presence of stated features, integers, steps or components but does not preclude the presence or addition of one or more other features, integers, steps, components or groups thereof. Thus, unless the context clearly requires otherwise, throughout the description and the claims, the words ‘comprise’, ‘comprising’, ‘includes’, ‘including’ and the like are to be construed in an inclusive sense as opposed to an exclusive or exhaustive sense; that is to say, in the sense of “including, but not limited to”.

1. An electronic lateral flow assay test reader for reading a lateral flow test strip, the electronic lateral flow assay test reader having a light guide comprising a window structure for framing a development area of the test strip, the development area comprising portions that include a test background region and at least one test result line, wherein the dimensions of the window structure are configured to maximise the proportion of the at least one test result line framed relative to the proportion of test background region framed.

2. An electronic reader as claimed in claim 1, wherein the window structure comprises individual windows for framing respective portions of the development area of the test strip such that any of the test background region framed by the window structure is minimised and wherein the respective portions of the development area of the test strip framed by the individual windows comprises one or more of: a test line; a control line.

3. An electronic reader as claimed in claim 1 wherein the test strip includes strip background and the window structure further comprises at least one window for framing strip background.

4. (canceled)

5. An electronic reader as claimed in claim 1, wherein the reader has a housing which retains reader components including:
the test strip;
a PCB incorporating test measurement components; and
the light guide as a separate element, wherein the light guide is disposed in close proximity to the test strip.

6. (canceled)

7. An electronic reader as claimed in claim 1, further comprising a carrier adapted to retain reader components including a removably insertable cassette adapted for containing the lateral flow test strip, wherein the window structure of the light guide is formed by one or a combination of: the carrier; the cassette.

8. An electronic reader as claimed in claim 7 wherein the electronic reader comprises a unitary housing.

9. (canceled)

10. An electronic reader as claimed in claim 1, further comprising:
illumination sources for illuminating the at least one test result line and the test background region of the development area of the lateral flow test strip, and;
measurement sensors for detecting light received from the at least one test result line;
wherein each respective illumination source is paired with each respective measurement sensor.

11. (canceled)

12. An electronic reader as claimed in claim 7, wherein the cassette comprises:
a recess for receiving and nesting the lateral flow test strip therewithin,
at least two or more windows for framing respective portions of the development area of the test strip, the dimensions of the windows being configured to maximise the proportion of at least one result line framed relative to the proportion of test background framed.

13. An electronic reader as claimed in claim 7, wherein surfaces of the cassette comprise minimally reflective material.

14. (canceled)

15. (canceled)

16. (canceled)

17. (canceled)

18. (canceled)

19. (canceled)

20. Electronic reader apparatus for a lateral flow assay test strip, the apparatus comprising:
a cassette comprising a recess for receiving and nesting the lateral flow assay test strip therewithin; and
a carrier accommodating the cassette for engagement with the reader;
wherein the cassette is removably retained within the reader by a retention mechanism formed by parts of one or a combination of the reader, the cassette and the carrier and the retention mechanism is adapted to align individual windows of one or a combination of the cassette and the carrier wherein the aligned windows frame respective portions of a development area of the test strip.

21. (canceled)

22. Apparatus as claimed in claim 2 wherein the retention mechanism comprises a releasable snap fit mechanism residing upon or within one or a combination of the reader, the cassette and the carrier including one or more of:
snap fingers for retaining the cassette in place within the reader, and;
biasing means which assists in releasing the cassette from the reader, which are adapted to work together to ensure that the cassette is positioned consistently and correctly in the reader.

23. Apparatus as claimed in claim 22 wherein the snap fingers reside on the cassette and the biasing means resides on the carrier or the reader.

24. Apparatus as claimed in claim 20, further comprising biasing means formed by parts of one or a combination of the reader, the cassette and a carrier accommodating the cassette for engagement with the reader that urge the cassette towards electronic components of the reader used for measuring.

25. Apparatus as claimed in claim 20 wherein the reader is adapted for multiuse and comprises a self-closing door that prevents contaminants from entering a cavity of the multiuse reader when a cassette is not installed in the multiuse reader.

26. Apparatus as claimed in claim 25 wherein the door acts to align the cassette within the reader.

27. (canceled)

28. (canceled)

29. Apparatus as claimed in claim 20 wherein the reader is operable with the cassette by one of:
a slide-on mechanism; or
a clip-on mechanism.

30. An electronic lateral flow assay test reader for reading a lateral flow test strip having a development area comprising a test background region and at least one test result line, the electronic lateral flow assay test reader comprising,
a cassette for retaining the lateral flow assay test strip and a carrier adapted to removably retain the cassette therein;
at least one illumination LED operably associated with the one or a combination of the cassette and the carrier for illuminating the test strip;
a light guide comprising a window structure of one or a combination of the cassette and the carrier wherein the light guide is adapted to prevent light emitted or reflected from outside a selected portion of the development area of the test strip being directed to a sensor.

31. (canceled)

32. (canceled)

33 (canceled)

34. An electronic reader as claimed in claim 30, wherein the light guide is further adapted to direct light emitted or reflected from the selected portion of the development area of the lateral flow assay test strip to a sensor such that the proportion of the at least one test result line relative to the proportion of test background region in the selected portion of the development area of the test strip is maximized.

35. (canceled)

36. (canceled)

37. (canceled)

38. An electronic lateral flow assay test reader for reading a lateral flow test strip having a development area, the development area comprising portions that include a test background region and at least one test result line, the electronic lateral flow assay test reader comprising:
a cassette for retaining the test strip and a carrier adapted to removably retain the cassette therein;
at least one illumination LED operably associated with one or a combination of the cassette and the carrier for illuminating the test strip, and;
a light guide comprising a window structure wherein the light guide is adapted to prevent light emitted or reflected from outside a selected portion of the development area of the test strip being directed to a sensor and, wherein the window structure is formed by:
one of the cassette or the carrier, or;
a combination of the cassette and the carrier so as to split the light guide between the cassette and the carrier.

39. (canceled)

40. (canceled)

41. (canceled)

42. (canceled)

43. (canceled)

44. A cassette adapted for use with an electronic lateral flow assay test reader, the cassette comprising,
a recess for receiving and/or nesting a lateral flow test strip,
at least one window for framing a development area of the test strip when nested in the recess, the dimensions of the window being configured to maximise the proportion of at least one test result line of the development area framed relative to the proportion of a test background region of the development area framed.

45. (canceled)

46. (canceled)

47. (canceled)

48 (canceled)

49. (canceled)

50. (canceled)

51. A cassette as claimed in claim 44, wherein the cassette is adapted for removable insertion into a carrier to form a light guide that is formed by a combination of the cassette, and the carrier so as to split the light guide between the cassette and the carrier.
